SEQUENCE LISTING (10 sequences)

SEQ ID NO: 1 - protein, 7 aa, Unknown
  Description of Unknown Nuclear localization signal sequence
PKKKRKV

SEQ ID NO: 2 - protein, 7 aa, Unknown
  Description of Unknown Nuclear localization signal sequence
PKKKRRV

SEQ ID NO: 3 - protein, 16 aa, Unknown
  Description of Unknown Nuclear localization signal sequence
KRPAATKKAGQAKKKK

SEQ ID NO: 4 - protein, 20 aa, Human immunodeficiency virus
GRKKRRQRRRPPQPKKKRKV

SEQ ID NO: 5 - protein, 19 aa, Hepatitis B virus
PLSSIFSRIGDPPKKKRKV

SEQ ID NO: 6 - protein, 24 aa, Artificial Sequence
  Description of Artificial Sequence Synthetic peptide
GALFLGWLGAAGSTMGAPKKKRKV

SEQ ID NO: 7 - protein, 27 aa, Artificial Sequence
  Description of Artificial Sequence Synthetic peptide
GALFLGFLGAAGSTMGAWSQPKKKRKV

SEQ ID NO: 8 - protein, 21 aa, Artificial Sequence
  Description of Artificial Sequence Synthetic peptide
KETWWETWWTEWSQPKKKRKV

SEQ ID NO: 9 - protein, 6 aa, Artificial Sequence
  Description of Artificial Sequence Synthetic 6xHis tag
HHHHHH

SEQ ID NO: 10 - protein, 9 aa, Unknown
  Description of Unknown ′LAGLIDADG′ family peptide motif sequence
LAGLIDADG

(19) United States
(12) Patent Application Publication
(10) Pub. No.: US 2019/0390241 A1
(43) Pub. Date: Dec. 26, 2019

(54) VIRAL RESISTANT CELLS AND CULTURE SYSTEMS

(71) Applicant: SIGMA-ALDRICH CO. LLC, St. Louis, MO (US)

(72) Inventors: Joaquina Mascarenhas, St. Louis, MO (US); Audrey Chang, Rockville, MD (US); Nikolay Korokhov, Rockville, MD (US); David Onions, Glasgow (GB); Henry George, Belleville, IL (US); Kevin J. Kayser, San Francisco, CA (US)

(21) Appl. No.: 16/480,588

(22) PCT Filed: Jan. 24, 2018
(86) PCT No.: PCT/US2018/015023

§ 371 Date: Jul. 24, 2019

Related U.S. Application Data
(60) Provisional application No. 62/449,691, filed on Jan. 24, 2017.

(51) Int. Cl. C12P 21/02; C12N 9/22

(52) U.S. Cl. CPC C12P 21/02; C12N 2510/02; C12N 9/22

(57) ABSTRACT

Mammalian cell lines genetically engineered to be viral resistant, cell culture systems comprising agents that inhibit viral entry into or translocation within cells, and methods of using said cell lines and/or said cell culture systems to reduce or prevent viral contamination of biologic production systems.

### CROSS-REFERENCE TO RELATED APPLICATION

The present application claims the benefit of priority of U.S. Provisional Patent Application No. 62/449,691, filed Jan. 24, 2017, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Jan. 23, 2018, is named SIG220PCT_SL.txt and is 3,293 bytes in size.

### FIELD OF THE INVENTION

The present disclosure relates to mammalian cell lines engineered to have viral resistance and cell culture systems comprising agents that inhibit viral entry into or translocation within cells.

### BACKGROUND

The use of recombinantly-produced therapeutic proteins for the treatment of many diseases or conditions such as cancer and autoimmune diseases continues to increase. However, large-scale production of these protein therapeutics still remains a challenge. For example, the commercial manufacturing process must deliver a reliably high-yield with downstream processes producing an extremely pure product allowing only trace amounts, to preferably, no contaminants.

The use of animal component-free media has significantly reduced the incidence of adventitious viral contamination. Additionally, the implementation of procedures such as ultrafiltration, high temperature short time processing, and/or UVC irradiation of bulk materials has further reduced the incidence of contamination. Nevertheless, the risk of viral contamination still remains. A contamination incident would be catastrophic for the manufacturer in terms of loss of product, temporary withdrawal for the market, and extensive decontamination costs. Thus, there is a need for mammalian cell lines and/or cell culture systems having increased resistance to viral infection.

### SUMMARY

Among the various aspects of the present disclosure is the provision of mammalian cell lines that are engineered to have reduced expression of a protein chosen from integrin, beta 1; integrin, beta 2; integrin, beta 3; integrin, beta 4; integrin, beta 5; integrin, beta 6; integrin, beta 7; integrin, beta 8; integrin, alpha 1; integrin, alpha 2; integrin, alpha 3; integrin, alpha 4; integrin, alpha 5; integrin, alpha 6; integrin, alpha 7; integrin, alpha 8; integrin, alpha 9; integrin, alpha 10; integrin, alpha 11, integrin, alpha D, integrin, alpha E; integrin, alpha L; integrin, alpha V; integrin, alpha 2B; integrin, alpha X; talin 1; talin 2; xylosyltransferase 1; xylosyltransferase 2; β4-galactosyltransferase; β3-galactosyltransferase; β3-GlcA transferase; exostosin 1; exostosin 2; exostosin-like 1; exostosin-like 2; exostosin-like 3; bifunctional heparan sulfate N-deacetylase/N-sulfotransferase 1; D-glucuronyl C5-epimerase; heparan sulfate 2-O-sulfotransferase 1; heparan sulfate 6-O-sulfotransferase 1; heparan sulfate 3-O sulfotransferase; carbohydrate (N-acetylgalactosamine 4-O) sulfotransferase 8; carbohydrate (chondroitin 6) sulfotransferase 3; carbohydrate (N-acetylglucosamine 6-O) sulfotransferase 7; carbohydrate (N-acetylgalactosamine 4-sulfate 6-O) sulfotransferase 15; carbohydrate (N-acetylglucosamine 6-O sulfotransferase 5; hyaluronan synthase protein 1; hyaluronan synthase protein 2; hyaluronan synthase protein 3; dynamin-1; dynamin-2; dynamin-3; caveolin 1; cell division cycle 42; ADP-ribosylation factor 6; Ras-related C3 botulinum toxin substrate 1; lysosome-associated membrane glycoprotein 1; chloride channel, voltage-sensitive 1; chloride channel, voltage-sensitive 2; H(+)/Cl(−) exchange transporter 3; H(+)/Cl(−) exchange transporter 4; H(+)/Cl(−) exchange transporter 5; H(+)/Cl(−) exchange transporter 6; H(+)/Cl(−) exchange transporter 7; V-type proton ATPase catalytic subunit A; ATPase, H+ transporting, lysosomal 70 kDa, V1 subunit B1; ATPase, H+ transporting, lysosomal 70 kDa, V1 subunit B2; ATPase, H+ transporting, lysosomal accessory Protein 1 (Ac45); ATPase, H+ transporting, lysosomal 42 kDa, V1 subunit C2; serine-protein kinase ATM; RAF proto-oncogene serine/threonine-protein kinase; ATM serine/threonine kinase; bromodomain adjacent to zinc finger domain 1B; casein kinase 2, alpha 1 polypeptide; casein kinase II subunit alpha; cofilin-1; cofilin-2; exportin-1; amyloid beta (A4) precursor protein-binding, family B, member 1 interacting protein; phosphatidylinositol-4-phosphate 5-kinase, type I, gamma, or combination thereof.

Another aspect of the present disclosure encompasses a cell culture system comprising a Selective Inhibitor of Nuclear Export (SINE), a sialic acid analog, a small molecule inhibitor of CMP sialic acid transporter, a sialidase, a neuraminidase, or combination thereof, and a cell growth medium.

Still another aspect of the present disclosure provides a method for reducing or preventing viral contamination of a recombinant protein product, the method comprising obtaining a viral resistant mammalian cell line as disclosed herein and/or a cell culture system as disclosed herein and expressing the recombinant protein product in the cell line and/or cell culture system.

Other aspects and iterations of the disclosure are described in more detail below.

### BRIEF DESCRIPTION OF THE DRAWINGS

FIG. 1 presents the resistance to MVM viral infection of dynamin double knockout CHO clones. Plotted is the number of viral genome copies per sample of the indicated cells at 0 and 21 hours post infection.

FIG. 2 shows the viral resistance of dynamin double or triple knockout CHO clones. Plotted is the percentage of viral genome copies relative to CHO wild type cells of the indicated cells at 0 and 21 hours post infection.

FIG. 3 presents the resistance to MVM viral infection of integrin beta 1 or integrin alpha 5 knockout CHO clones. Plotted is the number of viral genome copies per sample of the indicated cells at 0 and 21 hours post infection.

### DETAILED DESCRIPTION

The present disclosure provides mammalian cell lines engineered to hinder, inhibit, or prevent viral entry such that they exhibit viral resistance. The engineered cell lines having viral resistance are modified to have reduced or eliminated expression of proteins involved with viral entry into a cell, viral movement/translocation within the cell, and/or viral egress from the cell. The present disclosure also provides cell culture systems comprising reagents that hinder, inhibit, or prevent viral entry into or translocation within cells. Also provided are methods for using the cell lines and/or the culture systems disclosed herein for the production of recombinant proteins, wherein the recombinant protein products are essentially devoid of viral contamination. Use of the cell lines that are resistant to viral infection and/or cell culture systems that inhibit or prevent viral entry, therefore, reduces or eliminates the risk of viral contamination of biologic production systems and the resultant protein products.

### (I) Viral Resistant Cell Lines

One aspect of the present disclosure encompasses mammalian cell lines that are engineered to have viral resistance. Stated another way, the cell lines disclosed herein have increased resistance to infection by one or more viruses as compared to unmodified, parental cell lines. More specifically, entry of the virus and/or propagation of the virus is reduced or eliminated in the engineered cell lines disclosed herein as compared to unmodified parental cell lines.

### (a) Disrupted Expression

In some embodiments, the mammalian cell lines disclosed herein are modified/engineered to have reduced or no expression of one or more proteins involved in viral entry into the cell, viral movement/translocation within the cell, and/or viral egress from the cell. For example, the cell lines can have reduced or eliminated expression of cell surface receptors (e.g., integrins) that mediate viral cell attachment and internalization, reduced expression of proteins (e.g., gelsolin, talins) that regulate integrin receptor affinity and valency, reduced or no expression of enzymes/proteins involved in glycan biosynthesis, reduced or no expression of proteins (e.g., clathrin, caveolae, etc.) involved in viral entry mechanisms into cells, reduced or no expression of proteins involved in cytoplasmic trafficking of viruses through endosomes, reduced or no expression of protein involved in endosomal and lysosomal structure and function, reduced or no expression of proteins involved in proteasome interactions, and/or reduced or no expression of proteins involved with viral nuclear translocation (entry and/or exit). Specific proteins whose expression can be reduced or eliminated are listed in Table A.

TABLE A
Target Genes

| | | UniProtKB |
|---|---|---|
| Protein | Gene | (human) |
| Integrin, beta 1 | ITGB1 | P05556 |
| Integrin, beta 2 | ITGB2 | P05107 |
| Integrin, beta 3 | ITGB3 | Q16157 |
| Integrin, beta 4 | ITGB4 | P16144 |
| Integrin, beta 5 | ITGB5 | P18084 |
| Integrin, beta 6 | ITGB6 | P18564 |
| Integrin, beta 7 | ITGB7 | P26010 |
| Integrin, beta 8 | ITGB8 | P26012 |
| Integrin, alpha 1 | ITGA1 | P56199 |
| Integrin, alpha 2 | ITGA2 | P17301 |
| Integrin, alpha 3 | ITGA3 | P26006 |
| Integrin, alpha 4 | ITGA4 | P13612 |
| Integrin, alpha 5 | ITGA5 | P08548 |
| Integrin, alpha 6 | ITGA6 | P23229 |
| Integrin, alpha 7 | ITGA7 | Q13683 |
| Integrin, alpha 8 | ITGA8 | P52708 |
| Integrin, alpha 9 | ITGA9 | Q13797 |
| Integrin, alpha 10 | ITGA10 | O75578 |
| Integrin, alpha 11 | ITGA11 | Q9UKX5 |
| Integrin, alpha D | ITGAD | Q13349 |
| Integrin, alpha E | ITGAE | P38570 |
| Integrin, alpha L | ITGAL | P20701 |
| Integrin, alpha M | ITGAM | P11216 |
| Integrin, alpha V | ITGAV | P06756 |
| Integrin, alpha 2B | ITGA2B | P08514 |
| Integrin, alpha X | ITGAX | P20702 |
| Talin 1 | TLN1 | Q9Y490 |
| Talin 2 | TLN2 | Q9Y4G6 |
| Xylosyltransferase 1 | XYLT1 | Q86Y38 |
| Xylosyltransferase 2 | XYLT2 | Q9H1B5 |
| β4-Galactosyltransferase (GalT-I) | B4GALT1 | P15291 |
| β3-Galactosyl transferase(GalT-II) | β3GALT2 | O43825 |
| β3-GlcA transferase (GlcAT-I) | B3GAT3 | O94766 |
| Exostosin 1 | EXT1 | Q16394 |
| Exostosin 2 | EXT2 | Q93063 |
| Exostosin-like 1 | EXTL1 | Q92935 |
| Exostosin-like 2 | EXTL2 | Q05DH5 |
| Exostosin-like 3 | EXTL3 | O43909 |
| Bifunctional heparan | NDST1 | P52848 |
| sulfate N-deacetylase/ | | |
| N-sulfotransferase 1 | | |
| D-glucuronyl C5-epimerase | GLCE | O94923 |
| Heparan sulfate 2-O- | HS2ST1 | Q7LGA3 |
| sulfotransferase 1 | | |
| Heparan sulfate 6-O- | H6ST1 | O60243 |
| sulfotransferase 1 | | |
| Heparan sulfate 3-O | HS3ST1 | O14792 |
| sulfotransferases | | |
| Carbohydrate (N-acetylgalactosamine | CHST8 | Q9H2A9 |
| 4-0) sulfotransferase 8 | | |
| Carbohydrate (chondroitin 6) | CHST3 | Q7LGC8 |
| sulfotransferase 3 | | |
| Carbohydrate (N-acetylglucosamine | CHST7 | Q9NS84 |
| 6-O) sulfotransferase 7 | | |
| Carbohydrate (N-acetylgalactosamine | CHST15 | Q7LFX5 |
| 4-sulfate 6-O) sulfotransferase 15 | | |
| Carbohydrate (N-acetylglucosamine | CHST5 | Q9GZS9 |
| 6-O) sulfotransferase 5 | | |
| Hyaluronan synthase protein 1 | HAS1 | Q92839 |
| Hyaluronan synthase protein 2 | HAS2 | Q92819 |
| Hyaluronan synthase protein 3 | HAS3 | O00219 |
| Dynamin-1 | DNM1 | Q05193 |
| Dynamin-2 | DNM2 | P50570 |
| Dynamin-3 | DNM3 | Q9UQ16 |
| Caveolin 1 | CAV1 | Q03135 |
| Cell Division Cycle 42 | CDC42 | P60953 |
| ADP-Ribosylation Factor 6 | ARF6 | P62330 |
| Ras-Related C3 Botulinum Toxin | RAC1 | P63000 |
| Substrate 1 (Rho Family, Small | | |
| GTP Binding Protein Rac1) | | |
| Lysosome-associated membrane | LAMP1 | P11279 |
| glycoprotein 1 | | |
| Chloride channel, voltage-sensitive 1 | CLCN 1 | P35523 |
| Chloride channel, voltage-sensitive 2 | CLCN 2 | P51788 |
| H(+)/Cl(−) exchange transporter 3 | CLCN 3 | P51790 |
| H(+)/Cl(−) exchange transporter 4 | CLCN4 | P51793 |
| H(+)/Cl(−) exchange transporter 5 | CLCN5 | P51795 |
| H(+)/Cl(−) exchange transporter 6 | CLCN6 | P51797 |
| H(+)/Cl(−) exchange transporter 7 | CLCN7 | P51798 |
| V-type proton ATPase catalytic subunit A | ATP6V1A | P38606 |
| ATPase, H+ transporting, lysosomal 70 | Atp6v1 b1 | P15313 |
| kDa, V1 subunit B1 | | |
| ATPase, H+ transporting, lysosomal 70 | Atp6v1 b2 | P21281 |
| kDa, V1 subunit B2 | | |
| ATPase, H+ Transporting, Lysosomal | ATP6AP1 | Q15904 |
| Accessory Protein 1 (Ac45) | | |
| ATPase, H+ transporting, lysosomal 42 | ATP6V1C2 | Q8NEY4 |
| kDa, V1 subunit C2 | | |
| Serine-protein kinase ATM | ATM | Q13315 |
| RAF proto-oncogene serine/threonine- | Raf 1 | P04049 |
| protein kinase | | |
| ATM serine/threonine kinase | ATM | Q13315 |
| Bromodomain adjacent to zinc finger | BAZ1B | Q9UIG0 |
| domain 1B | | |
| Casein kinase 2, alpha 1 polypeptide | CSNK2A1 | Q5U5J2 |
| Casein kinase II subunit alpha | CSNK2A2 | P19784 |
| Cofilin-1 | CFL1 | P23528 |
| Cofilin-2 | CFL2 | Q9Y281 |
| Exportin-1 | XPO1 | O14980 |
| Amyloid Beta (A4) Precursor Protein- | APBB1IP | Q7Z5R6 |
| Binding, Family B, Member 1 | | |
| Interacting Protein (Rap1-guanosine | | |
| triphosphate-interacting adaptor molecule) | | |
| Phosphatidylinositol-4-Phosphate 5- | PIP5K1C | O60331 |
| Kinase, Type I, Gamma | | |

In general, reduced expression is due to modification of at least one nucleic acid sequence (i.e., chromosomal DNA or RNA transcript) encoding the protein of interest such that the cell line produces reduced levels (i.e., knocked down) of the encoded protein. In other embodiments, the mammalian cell lines are modified to inactivate (i.e., knockout) all the nucleic acid sequences encoding the protein of interest such that no protein product is produced.

In some embodiments, the cell line has reduced or eliminated expression of one protein listed in Table A. In other embodiments, the cell line has reduced or eliminated expression of two proteins listed in Table A. In further embodiments, the cell line has reduced or eliminated expression of three proteins listed in Table A. In still other embodiments, the cell line has reduced or eliminated expression of four proteins listed in Table A. In additional embodiments, the cell line has reduced or eliminated expression of five proteins listed in Table A. In further embodiments, the cell line has reduced or eliminated expression of six proteins listed in Table A. In yet other embodiments, the cell line has reduced or eliminated expression of seven or more proteins listed in Table A.

Expression of proteins of interest can be reduced or eliminated by genetically modifying chromosomal sequences encoding the proteins of interest. Chromosomal sequences of interest can be modified using targeted endonuclease-mediated genomic editing techniques, which are detailed below in section (V)(a). For example, chromosomal sequences can be modified to contain a deletion of at least one nucleotide, an insertion of at least one nucleotide, a substitution of at least one nucleotide, or a combination thereof, such that the reading frame is shifted and no protein product is produced (i.e., the chromosomal sequence is inactivated). In cases in which the locus of interest is biallelic and one chromosomal sequence is inactivated, the cell line produces reduced levels of the protein of interest (i.e., knocked down). In cases in which the locus of interest is biallelic and both chromosomal sequences are inactivated, the cell line produces no protein product (i.e., knocked out). In cases in which the locus of interest is monoallelic, inactivation of the single chromosomal sequence results in a knock out phenotype. Alternatively, the deletion(s), insertion(s), and/or substitution(s) in the modified chromosomal sequence can lead to the production of an altered protein product (e.g., truncated protein, protein with altered activity, affinity, etc.).

In still other embodiments, expression of the protein of interest can be reduced or eliminated using RNA interference-mediated mechanisms, which are described below in section (V)(b).

In some embodiments, the level of the protein(s) of interest can be reduced by at least about 5%, by at least about 20%, by at least about 50%, by at least about 80%, by at least about 90%, by at least about 95%, by at least about 99%, or more than about 99%. In other embodiments, the level of the protein of interest can be reduced to non-detectable levels using techniques standard in the field (e.g., Western immunoblotting assays, ELISA enzyme assays, and the like).

In general, resistance (or susceptibility) to viral infection can be determined by comparing the response of the engineered mammalian cell lines to exposure to a virus or viruses with the response of unmodified (non-engineered) parental cells to the same viral challenge. Viral infection of the cell line and/or viral propagation in the cell line can be analyzed by a variety of techniques. Non-limiting examples of suitable techniques include nucleic acid detection methods (e.g., Southern nucleic acid blotting assay to detect the presence of specific viral nucleic acids, PCR or RT-PCR to detect viral nucleic acids, sequencing methods, and the like), antibody-based techniques (e.g., Western immunoblotting techniques using anti-viral protein antibodies, ELISA methods, and so forth), bioassays, (e.g., plaque assays, cytopathic effect assays, and the like), and microscopic techniques (e.g., electron microscopy to detect viral particles, and so forth). In some embodiments, infection and/or propagation of the virus within the engineered mammalian cell lines can be reduced by at least about 10%, at least about 20%, at least about 40%, at least about 60%, at least about 80%, at least about 90%, at least about 95% at least about 99%, or more than about 99% relative to that of unmodified parental cells. In specific embodiments, the engineered mammalian cell lines are resistant to viral infection, i.e., the virus is unable to enter and/or propagate in the engineered mammalian cell lines.

### (b) Optional Additional Modifications

The mammalian cell lines disclosed herein can further comprise disrupted expression of one or more proteins involved in cellular processes related to viral entry/propagation and/or protein glycosylation processes, or can be modified to interfere with viral proteins.

(i) Interfere with Cellular Processes

In some embodiments, the cell line can be further modified/engineered to have reduced or eliminated expression of galectin-3, vimentin, caspase 3, gelsolin, WD repeat containing protein 1 (Wdr1), radixin, moesin, or combinations thereof.

In other embodiments, the cell can be further modified/engineered to have increased expression (i.e., overexpression) of an anti-viral protein such as, e.g., promyelocytic leukemia protein (PML or TRIM19). PML is a viral restriction factor that inhibits viral process ranging from viral uncoating to viral transcription. Increased expression can be achieved by introducing one or more copies of a nucleic acid sequence encoding the anti-viral protein of interest or by modifying endogenous chromosomal sequences. Additional copies of the sequence of interest can be integrated into the genome of the cell using targeted endonuclease-mediated genomic editing techniques, which are detailed below in section (V)(a). The additional copies can be placed under control of an endogenous promoter region or the additional copies can be linked to an exogenous promoter sequence prior to integration. Alternatively, additional copies of the sequence of interest (along with appropriate transcriptional control sequence) the anti-viral protein of interest can be can be extrachromosomal (e.g., episomal) for stable expression. In further embodiments, the cell lines can be genetically modified using targeted endonuclease-mediated editing techniques to modify transcriptional control regions (e.g., integrate additional or stronger promoter sequences or enhancer elements, and/or integrate epigenetic modifications) such that expression of the anti-viral protein of interest is increased. Expression of the anti-viral protein (e.g., PML) may be increased by at least about 5%, by at least about 20%, by at least about 50%, by at least about 2-fold, by at least about 4-fold, at least 10-fold, or more than 10-fold relative to unmodified cells.

In still other embodiments, the cell line can be further modified/engineered to have reduced or eliminated expression of enzymes or proteins involved in O-linked glycosylation. For example, the cell line can be deficient in core 1 elongation enzyme (also called core 1 synthase glycoprotein-N-acetylgalactosamine 3-beta-galactosyltransferase 1 or C1GalT1), core 1 enzyme chaperone (also called C1GalT1-specific chaperone or COSMC), or both. The deficiency can be due to inactivated chromosomal sequences encoding C1GalT1 and/or COSMC such that the cell line produces reduced levels or no C1GalT1 and/or COSMC protein.

In other embodiments, the cell line can be further modified/engineered to have reduced or eliminated expression of at least one sialyltransferase (ST). The sialyltransferase can be a sialyltransferase that adds sialic acid to galactose in an alpha-2,3 linkage conformation, a sialyltransferase that adds sialic acid to galactose or N-acetylgalactosamine in an alpha-2,6 linkage conformation, or a sialyltransferase that adds sialic acid to other sialic acid units in an alpha-2,8 linkage conformation. Non-limiting examples of suitable sialyltransferases include with St3 beta-galactoside alpha-2,3-sialyltransferase 1 (St3Gal1), St3 beta-galactoside alpha-2,3-sialyltransferase 2 (St3Gal2), St3 beta-galactoside alpha-2,3-sialyltransferase 3 (St3Gal3), St3 beta-galactoside alpha-2,3-sialyltransferase 4 (St3Gal4), St3 beta-galactoside alpha-2,3-sialyltransferase 5 (St3Gal5), St3 beta-galactoside alpha-2,3-sialyltransferase 6 (St3Gal6), St6 beta-galactosamide alpha-2,6-sialyltranferase 1 (St6Gal1), St6 beta-galactosamide alpha-2,6-sialyltranferase 2 (St6Gal2), St6 (alpha-N-acetyl-neuraminyl-2,3-beta-galactosyl-1,3)-N-acetylgalactosaminide 1 (St6GalNac1), St6 (alpha-N-acetyl-neuraminyl-2,3-beta-galactosyl-1,3)-N-acetylgalactosaminide 2 (St6GalNac2), St6 (alpha-N-acetyl-neuraminyl-2,3-beta-galactosyl-1,3)-N-acetylgalactosaminide 3 (St6GalNac3), St6 (alpha-N-acetyl-neuraminyl-2,3-beta-galactosyl-1,3)-N-acetylgalactosaminide 4 (ST6GalNac4), St6 (alpha-N-acetyl-neuraminyl-2,3-beta-galactosyl-1,3)-N-acetylgalactosaminide 5 (St6GalNac5), St6 (alpha-N-acetyl-neuraminyl-2,3-beta-galactosyl-1,3)-N-acetylgalactosaminide 6 (St6GalNac6), St8 alpha-N-acetyl-neuraminide alpha-2,8-sialyltransferase 1 (St8Sia1), St8 alpha-N-acetyl-neuraminide alpha-2,8-sialyltransferase 2 (St8Sia2), St8 alpha-N-acetyl-neuraminide alpha-2,8-sialyltransferase 3 (St8Sia3), St8 alpha-N-acetyl-neuraminide alpha-2,8-sialyltransferase 4 (St8Sia4), St8 alpha-N-acetyl-neuraminide alpha-2,8-sialyltransferase 5 (St8Sia5), or St8 alpha-N-acetyl-neuraminide alpha-2,8-sialyltransferase 6 (St8Sia6). The deficiency can be due to inactivated chromosomal sequences encoding the one or more sialyltransferase such that the cell line produces reduced levels or no protein product of the sialyltransferase of interest.

In further embodiments, the cell line can be further modified/engineered to have reduced or eliminated expression of at least one enzyme or protein involved in sialic acid synthesis or transport. Examples of enzymes or proteins involved in sialic acid synthesis or transport include, without limit, glucosamine (UDP-N-acetyl)-2-epimerase/N-acetylmannosamine kinase (GNE), N-acetylneuraminic acid synthase (NANS), N-acetylneuraminic acid phosphatase (NANP), cytidine monophosphate N-acetylneuraminic acid synthetase (CMAS), and cytidine monophosphate N-acetylneuraminic acid hydroxylase (CMAH), solute carrier family 35 (CMP-sialic acid transporter), member A1 (Slc35A1). The deficiency can be due to inactivated chromosomal sequences encoding the one or more proteins involved in sialic synthesis or transport such that the cell lines produces reduced levels or none of the protein of interest.

In additional embodiments, the cell line can be further modified/engineered to have reduced or eliminated expression of at least one enzyme or protein involved in N-glycosylation. In some instances, the enzyme or protein involved in N-glycosylation can be an N-acetylglucosylaminyltransferase, which adds a GlcNAc residue to a beta-linked mannose residue of an N-linked glycan. Examples include mannosyl (alpha-1,3-)-glycoprotein beta-1,2-N-acetylglucosaminyltransferase 1 (Mgat-1), mannosyl (alpha-1,6-)-glycoprotein beta-1,2-N-acetylglucosaminyltransferase 2 (Mgat-2), mannosyl (alpha-1,4-)-glycoprotein beta-1,4-N-acetylglucosaminyltransferase 3 (Mgat-3), mannosyl (alpha-1,3-)-glycoprotein beta-1,4-N-acetylglucosaminyltransferase 4 (Mgat-4), and mannosyl (alpha-1,6-)-glycoprotein beta-1,6-N-acetylglucosaminyltransferase 5 (Mgat-5). In other instances, the enzyme or protein involved in N-glycosylation can be a galactosyltransferase, which adds a galactose residue in a beta 1,4 linkage to a GlcNAc residue of an N-linked glycan. The galactosyltransferase, can be UDP-Gal:BetaGlcNAc beta 1,4-galactosyltransferase, polypeptide 1 (B4GalT1), UDP-Gal:BetaGlcNAc beta 1,4-galactosyltransferase, polypeptide 2 (B4GalT2), UDP-Gal:BetaGlcNAc beta 1,4-galactosyltransferase, polypeptide 3 (B4GalT3), UDP-Gal:BetaGlcNAc beta 1,4-galactosyltransferase, polypeptide 4 (B4GalT4), UDP-Gal:BetaGlcNAc beta 1,4-galactosyltransferase, polypeptide 5 (B4GalT5), UDP-Gal:BetaGlcNAc beta 1,4-galactosyltransferase, polypeptide 6 (B4GalT6), or UDP-Gal:BetaGlcNAc beta 1,4-galactosyltransferase, polypeptide 7 (B4GalT7). The deficiency can be due to inactivated chromosomal sequences encoding the one or more proteins involved in N-glycosylation such that the cell line produces reduced levels or none of the protein of interest.

Expression of the protein of interest can be modified using targeted endonuclease-mediated genomic editing techniques, which are detailed below in section (V)(a), or using RNA interference-mediated mechanisms, which are described below in section (V)(b).

(ii) Interfere with Viral Proteins

In other embodiments, the cell lines can be engineered to express molecules that inhibit or block viral replication and/or infectivity. For example, the cell lines can be engineered to stably express at least one RNA interference (RNAi) agent against specific viral proteins that are involved in replication and/or infectivity. Non-limiting examples of suitable viral proteins include nonstructural proteins such as NS1 or NS2, and capsid proteins such as VP1 or VP2. RNAi agents bind to target transcripts and prevent protein expression by mediating cleavage of the transcript cleavage or disrupting translation of the transcript.

In some embodiments, the RNAi agent can be a short interfering RNA (siRNA). In general, a siRNA comprises a double-stranded RNA molecule that ranges from about 15 to about 29 nucleotides in length, or more generally from about 19 to about 23 nucleotides in length. In specific embodiments, the siRNA can be about 21 nucleotides in length. The siRNA can optionally further comprise one or two single-stranded overhangs, e.g., a 3′ overhang on one or both ends. The siRNA can be formed from two RNA molecules that hybridize together or, alternatively, can be generated from a short hairpin RNA (shRNA) (see below). In some embodiments, the two strands of the siRNA can be completely complementary, such that no mismatches or bulges exist in the duplex formed between the two sequences. In other embodiments, the two strands of the siRNA can be substantially complementary, such that one or more mismatches and/or bulges exist in the duplex formed between the two sequences. In certain embodiments, one or both of the 5′ ends of the siRNA can have a phosphate group, while in other embodiments one or both of the 5′ ends can lack a phosphate group.

One strand of the siRNA, which is referred to as the “antisense strand” or “guide strand,” includes a portion that hybridizes with the target transcript. In some embodiments, the antisense strand of the siRNA can be completely complementary to a region of the target transcript, i.e., it hybridizes to the target transcript without a single mismatch or bulge throughout the length of the siRNA. In other embodiments, the antisense strand can be substantially complementary to the target region, i.e., one or more mismatches and/or bulges can exist in the duplex formed by the antisense strand and the target transcript. Typically, siRNAs are targeted to exonic sequences of the target transcript. Those of skill in the art are familiar with programs, algorithms, and/or commercial services that design siRNAs for target transcripts.

In other embodiments, the RNAi agent can be a short hairpin RNA (shRNA). In general, a shRNA is an RNA molecule comprising at least two complementary portions that are hybridized or are capable of hybridizing to form a double-stranded structure sufficiently long to mediate RNA interference (as described above), and at least one single-stranded portion that forms a loop connecting the regions of the shRNA that form the duplex. The structure can also be called a stem-loop structure, with the stem being the duplex portion. In some embodiments, the duplex portion of the structure can be completely complementary, such that no mismatches or bulges exist in the duplex region of the shRNA. In other embodiments, the duplex portion of the structure can be substantially complementary, such that one or more mismatches and/or bulges can exist in the duplex portion of the shRNA. The loop of the structure can be from about 1 to about 20 nucleotides in length, specifically from about 6 to about 9 nucleotides in length. The loop can be located at either the 5′ or 3′ end of the region that is complementary to the target transcript (i.e., the antisense portion of the shRNA).

The shRNA can further comprise an overhang on the 5′ or 3′ end. The optional overhang can be from about 1 to about 20 nucleotides in length, or more specifically from about 2 to about 15 nucleotides in length. In some embodiments, the overhang can comprise one or more U residues, e.g., between about 1 and about 5 U residues. In some embodiments, the 5′ end of the shRNA can have a phosphate group. In general, shRNAs are processed into siRNAs by the conserved cellular RNAi machinery. Thus, shRNAs are precursors of siRNAs and are similarly capable of inhibiting expression of a target transcript that is complementary of a portion of the shRNA (i.e., the antisense portion of the shRNA). Those of skill in the art are familiar with the available resources for the design and synthesis of shRNAs. An exemplary example is MISSION® shRNAs (Sigma-Aldrich).

The siRNA or shRNA can be expressed in vivo from an RNAi expression construct. Suitable constructs include plasmid vectors, phagemids, cosmids, artificial/mini-chromosomes, transposons, and viral vectors (e.g., lentiviral vectors, adeno-associated viral vectors, etc.). In one embodiment, the RNAi expression construct can be a plasmid vector (e.g., pUC, pBR322, pET, pBluescript, and variants thereof). The RNAi expression construct can comprise two promoter control sequences, wherein each is operably linked appropriate coding sequence such that two separate, complementary siRNA strands can be transcribed. The two promoter control sequences can be in the same orientation or in opposite orientations. In another embodiment, the RNAi expression vector can contain a promoter control sequence that drives transcription of a single RNA molecule comprising two complementary regions, such that the transcript forms a shRNA. In general, the promoter control sequence(s) will be RNA polymerase III (Pol III) promoters such as U6 or H1 promoters. In other embodiments, RNA polymerase II (Pol II) promoter control sequences can be used (some examples are presented below). The RNAi expression constructs can contain additional sequence elements, such as transcription termination sequences, selectable marker sequences, etc. The RNAi expression construct can be introduced into the cell line of interest using standard procedures. The RNAi expression construct can be chromosomally integrated in the cell line for stable expression. Alternatively, the RNAi expression construct can be extrachromosomal (e.g., episomal) in the cell line for stable expression.

In still other embodiments, the cell lines can be engineered to stably express at least one dominant negative form of a viral protein involved in replication and/or infectivity. A dominant negative form of a protein is altered or mutated such that it out competes or inhibits the wild type protein. Non-limiting examples of suitable proteins include viral nonstructural proteins such as NS1 or NS2, and viral capsid proteins such as VP1 or VP2. In specific embodiments, the cell line can be engineered to express a dominant negative form of one or more NS1 proteins.

A dominant negative protein can have a deletion, an insertion, and/or a substitution relative to the wild type protein (Lagna et al., 1998, Curr. Topics Dev. Biol, 36:75-98). The deletion, insertion, and/or substitution can be at the N-terminal, C-terminal, or an internal location in the protein. Means for generating mutant proteins (via site-directed mutagenesis, PCR-based mutagenesis, random mutagenesis, etc.) are well known in the art, as are means for identifying those having dominant negative effects. Cell lines can be transfected with expression construct(s) comprising sequence encoding the dominant negative protein(s), wherein the coding sequence is operably linked to a Pol II promoter control sequence for expression. The promoter control sequence can be constitutive, regulated, or tissue-specific.

Suitable constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable regulated promoter control sequences include without limit those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter. The promoter sequence can be wild type or it can be modified for more efficient or efficacious expression.

The expression construct can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

### (c) Cell Types

The viral resistant cell lines disclosed herein are mammalian cell lines. In some embodiments, the cell lines having resistance to viral infection can be derived from Chinese hamster ovary (CHO) cells; mouse myeloma NS0 cells; baby hamster kidney (BHK) cells; mouse embryonic fibroblast 3T3 cells (NIH3T3); mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, or human K562 cells. An extensive list of mammalian cell lines may be found in the American Type Culture Collection catalog (ATCC, Manassas, Va.). In other embodiments, the cell lines with viral resistance are non-human, mammalian cell lines. In further embodiments, the cell lines disclosed herein are other than mouse cell lines. In certain embodiments, the cell lines with viral resistance are CHO cell lines. Numerous CHO cell lines are available from ATCC. Suitable CHO cell lines include, but are not limited to, CHO-K1 cells and derivatives thereof.

In various embodiments, the cell lines can be deficient in glutamine synthase (GS), dihydrofolate reductase (DHFR), hypoxanthine-guanine phosphoribosyltransferase (HPRT), or a combination thereof. For example, the chromosomal sequences encoding GS, DHFR, and/or HPRT can be inactivated. In specific embodiments, all chromosomal sequences encoding GS, DHFR, and/or HPRT are inactivated in the cell lines.

### (d) Viruses

The engineered mammalian cell lines having viral resistance can be resistant to a variety of mammalian viruses. The virus can be a DNA virus or an RNA virus, and the virus can be enveloped or non-enveloped (“naked”). Non-limiting examples of suitable viruses include members of Parvoviridae, Reoviridae, Caliciviridae, Paramyxoviridae, Coronaviridae, Picornaviridae, Polyoma viridae, Bunyaviridae, or combination thereof. In some embodiments, the engineered mammalian cell lines are resistant to infection by at least one parvovirus. Non-limiting examples of suitable parvoviruses include minute virus of mouse (MVM) (which is also known as mouse minute virus (MMV) or rodent protoparvovirus 1), mouse parvovirus type-1 (MPV-1), mouse parvovirus type-2 (MPV-2), mouse parvovirus type-3 (MPV-3), porcine parvovirus 1, bovine parovirus 1, and human parvovirus (e.g., human parovirus B19, human parovirus 4, human parovirus 5, etc.). In particular embodiments, the parvovirus can be MVM. In other embodiments, the virus can be a reovirus, such as mammalian reovirus-3, mammalian orthoreovirus, avian orthoreovirus, and the like). In specific embodiments, the genetically modified mammalian cell lines are resistant to MVM infection.

In some embodiments, the engineered mammalian cell lines having resistance to viral infection can also have resistance to infection by organisms in the order Mollicutes. In particular, the cell lines disclosed herein can be resistant to infection by the genera mycoplasma or spiroplasma.

### (e) Optional Nucleic Acid Encoding Recombinant Protein

In some embodiments, the mammalian cell lines having resistance to viral infection can further comprise at least one nucleic acid encoding a recombinant protein. In general, the recombinant protein is heterologous, meaning that the protein is not native to the cell. The recombinant protein may be, without limit, a therapeutic protein chosen from an antibody, a fragment of an antibody, a monoclonal antibody, a humanized antibody, a humanized monoclonal antibody, a chimeric antibody, an IgG molecule, an IgG heavy chain, an IgG light chain, an IgA molecule, an IgD molecule, an IgE molecule, an IgM molecule, a vaccine, a growth factor, a cytokine, an interferon, an interleukin, a hormone, a clotting (or coagulation) factor, a blood component, an enzyme, a therapeutic protein, a nutraceutical protein, a functional fragment or functional variant of any of the forgoing, or a fusion protein comprising any of the foregoing proteins and/or functional fragments or variants thereof.

In some embodiments, the nucleic acid encoding the recombinant protein can be linked to sequence encoding hypoxanthine-guanine phosphoribosyltransferase (HPRT), dihydrofolate reductase (DHFR), and/or glutamine synthase (GS), such that HPRT, DHFR, and/or GS may be used as an amplifiable selectable marker. The nucleic acid encoding the recombinant protein also can be linked to sequence encoding at least one antibiotic resistance gene and/or sequence encoding marker proteins such as fluorescent proteins. In some embodiments, the nucleic acid encoding the recombinant protein can be part of an expression construct. As detailed elsewhere expression constructs or vectors can comprise additional expression control sequences (e.g., enhancer sequences, Kozak sequences, polyadenylation sequences, transcriptional termination sequences, etc.), selectable marker sequences, origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

In further embodiments, the nucleic acid encoding the recombinant protein can be located extrachromosomally. That is, the nucleic acid encoding the recombinant protein can be transiently expressed from a plasmid, a cosmid, an artificial chromosome, a minichromosome, or another extrachromsomal construct. In other embodiments, the nucleic acid encoding the recombinant protein can be chromosomally integrated into the genome of the cell. The integration can be random or targeted. Accordingly, the recombinant protein can be stably expressed. In some iterations of this embodiment, the nucleic acid sequence encoding the recombinant protein can be operably linked to an appropriate heterologous expression control sequence (i.e., promoter). In other iterations, the nucleic acid sequence encoding the recombinant protein can be placed under control of an endogenous expression control sequence. The nucleic acid sequence encoding the recombinant protein can be integrated into the genome of the cell line using homologous recombination, targeting endonuclease-mediated genome editing, viral vectors, transposons, plasmids, and other well-known means. Additional guidance can be found in Ausubel et al. 2003, supra and Sambrook & Russell, 2001, supra.

### (f) Exemplary Embodiments

In specific embodiments, the mammalian cell lines having viral resistance are CHO cell lines. The viral resistant CHO cell lines can be resistant to infection by minute virus of mouse (MVM) (which is also known as mouse minute virus (MMV) or rodent protoparvovirus 1) and/or mammalian reovirus 3. Specifically, the genetically modified CHO cell lines have increased resistance to MVM or reovirus-3 infection as compared to unmodified parental CHO cell lines. In some embodiments, the unmodified parental cell line is a CHO (GS −/−) cell line. In particular, the viral resistant CHO cell lines have reduced or eliminated expression of integrin, beta 1; integrin, beta 2; integrin, beta 3; integrin, beta 4; integrin, beta 5; integrin, beta 6; integrin, beta 7; integrin, beta 8; talin 1; talin 2; xylosyltransferase 1; xylosyltransferase 2; β4-galactosyltransferase; β3-galactosyltransferase; β3-GlcA transferase; exostosin 1; exostosin 2; exostosin-like 1; exostosin-like 2; exostosin-like 3; bifunctional heparan sulfate N-deacetylase/N-sulfotransferase 1; D-glucuronyl C5-epimerase; heparan sulfate 2-O-sulfotransferase 1; heparan sulfate 6-O-sulfotransferase 1; heparan sulfate 3-O sulfotransferase; carbohydrate (N-acetylgalactosamine 4-O) sulfotransferase 8; carbohydrate (chondroitin 6) sulfotransferase 3; carbohydrate (N-acetylglucosamine 6-O) sulfotransferase 7; carbohydrate (N-acetylgalactosamine 4-sulfate 6-O) sulfotransferase 15; carbohydrate (N-acetylglucosamine 6-O sulfotransferase 5; hyaluronan synthase protein 1; hyaluronan synthase protein 2; hyaluronan synthase protein 3; dynamin-1; dynamin-2; dynamin-3; caveolin 1; cell division cycle 42; ADP-ribosylation factor 6; Ras-related C3 botulinum toxin substrate 1; lysosome-associated membrane glycoprotein 1; chloride channel, voltage-sensitive 1; chloride channel, voltage-sensitive 2; H(+)/Cl(−) exchange transporter 3; H(+)/Cl(−) exchange transporter 4; H(+)/Cl(−) exchange transporter 5; H(+)/Cl(−) exchange transporter 6; H(+)/Cl(−) exchange transporter 7; V-type proton ATPase catalytic subunit A; ATPase, H+ transporting, lysosomal 70 kDa, V1 subunit B1; ATPase, H+ transporting, lysosomal 70 kDa, V1 subunit B2; ATPase, H+ transporting, lysosomal accessory Protein 1 (Ac45); ATPase, H+ transporting, lysosomal 42 kDa, V1 subunit C2; serine-protein kinase ATM; RAF proto-oncogene serine/threonine-protein kinase; ATM serine/threonine kinase; bromodomain adjacent to zinc finger domain 1B; casein kinase 2, alpha 1 polypeptide; casein kinase II subunit alpha; cofilin-1; cofilin-2; exportin-1; amyloid beta (A4) precursor protein-binding, family B, member 1 interacting protein; phosphatidylinositol-4-phosphate 5-kinase, type I, gamma, or combination thereof.

### (II) Cell Culture System

Another aspect of the present disclosure provides a cell culture system comprising at least one agent that inhibits viral entry into or translocation within a cell grown in the culture system. Suitable agents include selective inhibitors of nuclear export (SINEs), sialic acid analogs, small molecule inhibitors of CMP sialic acid transporter, enzymes such as sialidases or neuraminidases, or combinations thereof.

In some embodiments, the cell culture system can comprise a SINE, which is a small molecule that generally binds to the cysteine residue (Cys528) in the NES binding groove of exportin (CRM1/XPO1). This binding irreversibly inactivates exportin (CRM1/XPO1). Non-limiting examples of suitable SINEs include leptomycin B, ratjadone, goniothalam in, N-azolylacrylates, anguinomycin, CBS9106, selinexor (KPT-330), verdinexor (KPT-335), KPT-185, KPT-251, KPT-276, or combination thereof.

In other embodiments, the cell culture system can comprise a sialic acid analog. Sialic acid analogs may interfere with the cellular sialic acid synthesis machinery such that cell surface sialic acid content is decreased. Additionally, neuraminic acid (a sialic acid derivative) analogs may mimic a cellular receptor and bind specific viruses with high affinity, thereby blocking attachment and infection by the virus. Suitable analogs of sialic acid or sialic acid derivatives include, without limit, P-3Fₐₓ-Neu5AC, oseltamivir, zanamivir, 5-acteylneuraminic acid derivates, 2-alpha-O-methyl-5-acetylneuraminic acid, or combinations thereof.

In still other embodiments, the cell culture system can comprise a small molecule inhibitor of CMP sialic acid transporter (i.e., SLC35A1). Inhibition of CMP sialic acid transport into the endoplasmic reticulum and Golgi vesicles can lead to the reduction of sialic acid on the surface of cells, thereby reducing viral entry. Non-limiting examples of suitable inhibitors of CMP sialic acid transporter include KI-8110, 2′-O-methyl CMP, 5-methyl CMP, or combinations thereof.

In additional embodiments, the cell culture system can comprise a sialidase, a neuraminidase, or combination thereof. Sialidases hydrolyze terminal sialic acid residues in oligosaccharides, glycoproteins, and glycolipids. Neuraminidases are glycoside hydrolase enzymes that cleave the glycosidic linkages of neuraminic acids. Thus, either can be used t sialidase, a neuraminidase, o remove sialic acid from the surface of cells, thereby reducing viral entry. The sialidase or neuraminidase can be derived from eukaryotic or prokaryotic cells. For example, the enzyme can be from Clostridium perfringens, Arthrobacter ureafaciens, Streptococcus pneumonia, or Vibrio cholera.

The amount of SINE, sialic acid analog, small molecule inhibitor of CMP sialic acid transporter, sialidase, or neuraminidase included in the cell culture system can vary. In general, the cell culture system contains an effective concentration of the compound (i.e., an amount sufficient to exert the intended effect). Those skilled in the art are familiar with means for determining the effective concentration of the above described compounds.

The cell culture system also comprises a cell growth medium. Non-limiting examples of suitable cell growth media include Dulbecco's Modified Eagle Medium (DMEM), F10 Nutrient Mixture, DMEM/F10, Ham's F12 Nutrient Mixture, Media 199, Minimum Essential Media (MEM), RPMI Medium 1640, Iscoe's Modified Dulbecco's Medium, specially serum free, animal component free media (e.g., CHO media, hybridoma media, insect media, vaccine media, etc.), Ames' Media, BGJb Medium, Click's Medium, SMRL-1066 Medium, Fischer's Medium, L-15 Medium, McCoy's 5A Modified Medium, NCTC Medium, Swim's S-77 Medium, Waymouth Medium, William's Medium E, and the like. In some instances, the cell growth medium is animal component free.

### (III) Compositions

Also provided herein are compositions comprising a mammalian cell line engineered to exhibit viral resistance, as described above in section (I), and at least one virus, wherein entry and/or propagation of the virus is reduced or eliminated in the engineered mammalian cell line. Thus, the cells in the composition are able to propagate, but the virus in the composition is unable to propagate because its entry into and/or replication within the cells is reduced or eliminated. The composition can further comprise a cell culture system as described above in section (II).

### (IV) Methods for Reducing or Preventing Viral Contamination

Another aspect of the present disclosure encompasses methods for reducing or preventing viral contamination of a recombinant protein product, or reducing the risk of viral contamination of a biologic production system. In general, the methods comprise providing engineered mammalian cell lines in which entry and/or propagation of at least one the virus is reduced or eliminated, which are described in section (I), and/or cell culture systems comprising agents that inhibit viral entry and/or propagation, which are described in section (II). The methods further comprise using said cell lines and/or cell culture systems for production of recombinant proteins having reduced or no viral contamination as compared to recombinant proteins prepared using unmodified parental cell lines and/or unmodified cell culture systems. The engineered mammalian cell lines exhibit resistant to viruses described in section (I)(d). Suitable recombinant proteins are described in section (I)(e). Means for producing or manufacturing recombinant proteins are well known in the field (see, e.g., “Biopharmaceutical Production Technology”, Subramanian (ed), 2012, Wiley-VCH; ISBN: 978-3-527-33029-4). In specific embodiments, the engineered mammalian cell lines are genetically modified to comprise at least one modified (or inactivated) chromosomal sequence such that the cell line is resistant to viral infection.

In general, the use of the engineered mammalian cell lines and/or cell culture systems disclosed herein reduces the ability of viruses to replicate in a fermenter or other bioproduction vessel such that the level of replicatable virus is at trace level or, ideally, at a level that is not detectable by industry standard best practices. Suitable methods include nucleic acid detection methods (e.g., Southern blotting to detect viral nucleic acids, PCR or RT-PCR to detect viral nucleic acids, sequencing methods, and the like), antibody-based techniques (e.g., Western immunoblotting using anti-viral protein antibodies, ELISA methods, and so forth), and microscopic techniques (e.g., cytopathic effect assays, electron microscopy to detect viral particles, etc.).

### (V) Methods for Preparing Viral Resistant Cell Lines

Yet another aspect of the present disclosure provides methods for engineering mammalian cell lines in which viral entry and/or propagation is reduced or eliminated. The engineered cell lines have reduced or eliminated expression of proteins involved in viral entry and/or propagation, as detailed above in section (I)(a). In some embodiments, the cells can have additional modifications, as described above I section (I)(b). Chromosomal sequences encoding proteins of interest can be knocked-down or knocked-out using a variety of techniques to generate the viral resistant cell lines. In some embodiments, the viral resistant cell lines can be prepared by a targeting endonuclease-mediated genome modification process. In other embodiments, the viral resistant cell lines can be prepared by RNA interference-mediated mechanisms. In still other embodiments, the viral resistant cell lines can be prepared by site-specific recombination systems, random mutagenesis, or other methods known in the art.

(a) Targeting Endonuclease-Mediated Genome Editing

Targeting endonucleases can be used to modify specific chromosomal sequences of interest. A specific chromosomal sequence can be inactivated by introducing into a cell a targeting endonuclease or a nucleic encoding the targeting endonuclease, which targets a specific chromosomal sequence. In one embodiment, the targeting endonuclease recognizes and binds the specific chromosomal sequence and introduces a double-stranded break that is repaired by a non-homologous end-joining (NHEJ) repair process. Because NHEJ is error prone, a deletion, insertion, and/or substitution of at least one nucleotide may occur, thereby disrupting the reading frame of the chromosomal sequence such that no protein product is produced. In another embodiment, the targeting endonucleases can also be used to alter a chromosomal sequence via a homologous recombination reaction by co-introducing a polynucleotide having substantial sequence identity with a portion of the targeted chromosomal sequence. The double-stranded break introduced by the targeting endonuclease is repaired by a homology-directed repair process such that the chromosomal sequence is exchanged with the polynucleotide in a manner that results in the chromosomal sequence being changed or altered (e.g., by integration of an exogenous sequence).

A variety of targeting endonucleases can be used to modify the chromosomal sequence(s) of interest. The targeting endonuclease can be a naturally-occurring protein or an engineered protein. Suitable targeting endonucleases include, without limit, zinc finger nucleases (ZFNs), CRISPR/Cas endonucleases, transcription activator-like effector (TALE) nucleases (TALENs), meganucleases, chimeric nucleases, site-specific endonucleases, and artificial targeted DNA double strand break inducing agents.

(i) Zinc Finger Nucleases

In specific embodiments, the targeting endonuclease can be a zinc finger nuclease (ZFN). ZFNs bind to a specific targeted sequence and introduce a double-stranded break into the targeted sequence. Typically, a ZFN comprises a DNA binding domain (i.e., zinc fingers) and a cleavage domain (i.e., nuclease), each of which is described below.

DNA Binding Domain.

A DNA binding domains or the zinc fingers can be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 can be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences as well as designing zinc finger binding domains are known in the art. For example, tools for identifying potential target sites in DNA sequences can be found at www.zincfingertools.org. Tools for designing zinc finger binding domains can be found at zifit.partners.org/ZiFiT. (See also, Mandell et al. (2006) Nuc. Acid Res. 34:W516-W523; Sander et al. (2007) Nuc. Acid Res. 35:W599-W605.)

A zinc finger binding domain can be designed to recognize and bind a DNA sequence ranging from about 3 nucleotides to about 21 nucleotides in length. In one embodiment, the zinc finger binding domain can be designed to recognize and bind a DNA sequence ranging from about 9 to about 18 nucleotides in length. In general, the zinc finger binding domains of the zinc finger nucleases used herein comprise at least three zinc finger recognition regions or zinc fingers, wherein each zinc finger binds 3 nucleotides. In one embodiment, the zinc finger binding domain comprises four zinc finger recognition regions. In another embodiment, the zinc finger binding domain comprises five zinc finger recognition regions. In still another embodiment, the zinc finger binding domain comprises six zinc finger recognition regions. A zinc finger binding domain can be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region include phage display and two-hybrid systems, which are described in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227, the entire disclosure of which is incorporated herein by reference.

Zinc finger binding domains and methods for design and construction of fusion proteins (and polynucleotides encoding same) are known to those of skill in the art and are described in detail in, for example, U.S. Pat. No. 7,888,121, which is incorporated by reference herein in its entirety. Zinc finger recognition regions and/or multi-fingered zinc finger proteins can be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

Cleavage Domain.

A zinc finger nuclease also includes a cleavage domain. The cleavage domain portion of the zinc finger nuclease can be obtained from any endonuclease or exonuclease. Non-limiting examples of endonucleases from which a cleavage domain can be derived include, but are not limited to, restriction endonucleases and homing endonucleases. See, for example, New England Biolabs Catalog or Belfort et al. (1997) Nucleic Acids Res. 25:3379-3388. Additional enzymes that cleave DNA are known (e.g., 51 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) can be used as a source of cleavage domains.

A cleavage domain also can be derived from an enzyme or portion thereof, as described above, that requires dimerization for cleavage activity. Two zinc finger nucleases can be required for cleavage, as each nuclease comprises a monomer of the active enzyme dimer. Alternatively, a single zinc finger nuclease can comprise both monomers to create an active enzyme dimer. As used herein, an “active enzyme dimer” is an enzyme dimer capable of cleaving a nucleic acid molecule. The two cleavage monomers can be derived from the same endonuclease (or functional fragments thereof), or each monomer can be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites can be separated by about 5 to about 18 nucleotides. For instance, the near edges can be separated by about 5, 6, 7, 8, 9, 10, 11, 12, 13, 14, 15, 16, 17 or 18 nucleotides. It will however be understood that any integral number of nucleotides or nucleotide pairs can intervene between two recognition sites (e.g., from about 2 to about 50 nucleotide pairs or more). The near edges of the recognition sites of the zinc finger nucleases, such as for example those described in detail herein, can be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fokl catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31978-31982. Thus, a zinc finger nuclease can comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fokl. This particular enzyme is active as a dimer (Bitinaite et al. (1998) Proc. Natl. Acad. Sci. USA 95: 10, 570-10, 575). Accordingly, for the purposes of the present disclosure, the portion of the Fokl enzyme used in a zinc finger nuclease is considered a cleavage monomer. Thus, for targeted double-stranded cleavage using a Fokl cleavage domain, two zinc finger nucleases, each comprising a Fokl cleavage monomer, can be used to reconstitute an active enzyme dimer. Alternatively, a single polypeptide molecule containing a zinc finger binding domain and two Fokl cleavage monomers can also be used.

In certain embodiments, the cleavage domain comprises one or more engineered cleavage monomers that minimize or prevent homodimerization. By way of non-limiting example, amino acid residues at positions 446, 447, 479, 483, 484, 486, 487, 490, 491, 496, 498, 499, 500, 531, 534, 537, and 538 of Fokl are all targets for influencing dimerization of the Fokl cleavage half-domains. Exemplary engineered cleavage monomers of Fokl that form obligate heterodimers include a pair in which a first cleavage monomer includes mutations at amino acid residue positions 490 and 538 of Fokl and a second cleavage monomer that includes mutations at amino-acid residue positions 486 and 499.

Thus, in one embodiment of the engineered cleavage monomers, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers can be prepared by mutating positions 490 from E to K and 538 from I to K in one cleavage monomer to produce an engineered cleavage monomer designated “E490K:I538K” and by mutating positions 486 from Q to E and 499 from I to K in another cleavage monomer to produce an engineered cleavage monomer designated “Q486E:I499K.” The above described engineered cleavage monomers are obligate heterodimer mutants in which aberrant cleavage is minimized or abolished. Engineered cleavage monomers can be prepared using a suitable method, for example, by site-directed mutagenesis of wild-type cleavage monomers (Fokl) as described in U.S. Pat. No. 7,888,121, which is incorporated herein in its entirety.

Additional domains. In some embodiments, the zinc finger nuclease further comprises at least one nuclear localization sequence (NLS). A NLS is an amino acid sequence which facilitates targeting the zinc finger nuclease protein into the nucleus to introduce a double stranded break at the target sequence in the chromosome. Nuclear localization signals are known in the art (see, e.g., Lange et al., J. Biol. Chem., 2007, 282:5101-5105). For example, in one embodiment, the NLS can be a monopartite sequence, such as PKKKRKV (SEQ ID NO: 1) or PKKKRRV (SEQ ID NO: 2). In another embodiment, the NLS can be a bipartite sequence. In still another embodiment, the NLS can be KRPAATKKAGQAKKKK (SEQ ID NO: 3). The NLS can be located at the N-terminus, the C-terminus, or in an internal location of the protein.

In additional embodiments, the zinc finger nuclease can also comprise at least one cell-penetrating domain. In one embodiment, the cell-penetrating domain can be a cell-penetrating peptide sequence derived from the HIV-1 TAT protein. As an example, the TAT cell-penetrating sequence can be GRKKRRQRRRPPQPKKKRKV (SEQ ID NO:4). In another embodiment, the cell-penetrating domain can be TLM (PLSSIFSRIGDPPKKKRKV; SEQ ID NO: 5), a cell-penetrating peptide sequence derived from the human hepatitis B virus. In still another embodiment, the cell-penetrating domain can be MPG (GALFLGWLGAAGSTMGAPKKKRKV; SEQ ID NO: 6 or GALFLGFLGAAGSTMGAWSQPKKKRKV; SEQ ID NO: 7). In an additional embodiment, the cell-penetrating domain can be Pep-1 (KETWWETWWTEWSQPKKKRKV; SEQ ID NO: 8), VP22, a cell penetrating peptide from Herpes simplex virus, or a polyarginine peptide sequence. The cell-penetrating domain can be located at the N-terminus, the C-terminus, or in an internal location of the zinc finger nuclease.

In still other embodiments, the zinc finger nuclease can further comprise at least one marker domain. Non-limiting examples of marker domains include fluorescent proteins, purification tags, and epitope tags. In one embodiment, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalamal, GFPuv, Sapphire, T-sapphire), cyan fluorescent proteins (e.g. ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In another embodiment, the marker domain can be a purification tag and/or an epitope tag. Suitable tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, 51, T7, V5, VSV-G, 6×His, biotin carboxyl carrier protein (BCCP), and calmodulin. The marker domain can be located at the N-terminus, the C-terminus, or in an internal location of the zinc finger nuclease.

The marker domain can be linked to the zinc finger nuclease by a 2A peptide (Szymczak et al., 2004, Nat. Biotechnol., 589(5):589-94). The 2A peptide was originally characterized in positive-strand RNA viruses, which produce a polyprotein that is “cleaved” during translation into mature individual proteins. More specifically, the 2A peptide region (˜20 amino acids) mediates “cleavage” at its own C-terminus to release itself from the downstream region of the polyprotein. In general, a 2A peptide sequence terminates with a glycine and a proline residue. During translation of a 2A peptide, the ribosome pauses after the glycine residue, resulting in release of the nascent polypeptide chain. Translation resumes, with the proline residue of the 2A sequence becoming the first amino acid of the downstream protein.

(ii) CRISPR/Cas Endonucleases

In other embodiments, the targeting endonuclease can be a CRISPR/Cas endonuclease. CRISPR/Cas endonucleases are RNA-guided endonucleases derived from CRISPR/Cas systems. Bacteria and archaea have evolved an RNA-based adaptive immune system that uses CRISPR (clustered regularly interspersed short palindromic repeat) and Cas (CRISPR-associated) proteins to detect and destroy invading viruses or plasmids. CRISPR/Cas endonucleases can be programmed to introduce targeted site-specific double-strand breaks by providing target-specific synthetic guide RNAs (Jinek et al., 2012, Science, 337:816-821).

Endonuclease. The CRISPR/Cas endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3,Csx17, Csx14, Csx10, Csx16, CsaX, Csx3, Csz1, Csx15, Csf1, Csf2, Csf3, Csf4, Cu1966, Cpf1, or derivatives thereof. In some embodiments, the RNA-guided endonuclease can be derived from a Cpf1 protein (Zetsche et al., Cell, 2015, 163: 759-771). In specific embodiments, the RNA-guided endonuclease is derived from a type II system Cas9 protein.

The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In one specific embodiment, the Cas9 protein is from Streptococcus pyogenes.

In general, CRISPR/Cas proteins comprise at least one RNA recognition and/or RNA binding domain. RNA recognition and/or RNA binding domains interact with the guide RNA such that the CRISPR/Cas protein is directed to a specific chromosomal or chromosomal sequence (i.e., target site). CRISPR/Cas proteins can also comprise nuclease domains (i.e., DNase or RNase domains), DNA binding domains, helicase domains, protein-protein interaction domains, dimerization domains, as well as other domains.

The CRISPR/Cas endonuclease can be derived from a wild type CRISPR/Cas protein, a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, nuclease (i.e., DNase, RNase) domains of the CRISPR/Cas protein can be modified, deleted, or inactivated. The CRISPR/Cas protein can be truncated to remove domains that are not essential for the function of the protein. The CRISPR/Cas protein also can be truncated or modified to optimize the activity of the protein or an effector domain fused with the CRISPR/Cas protein.

In some embodiments, the CRISPR/Cas endonuclease can be derived from a wild type Cas9 protein or fragment thereof. In other embodiments, the CRISPR/Cas endonuclease can be derived from a modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-strand break in DNA (Jinek et al., 2013, Science, 337: 816-821). In one embodiment, the CRISPR-based endonuclease is derived from a Cas9 protein and comprises two function nuclease domains.

The target sites recognized by naturally occurring CRISPR/Cas systems typically having lengths of about 14-15 bp (Cong et al., 2013, Science, 339:819-823). The target site has no sequence limitation except that sequence complementary to the 5′ end of the guide RNA (i.e., called a protospacer sequence) is immediately followed by (3′ or downstream) a consensus sequence. This consensus sequence is also known as a protospacer adjacent motif (or PAM). Examples of PAM for Cas9 based systems include, but are not limited to, NGG, NGGNG, NNAGAAW, NNGRRN, NNNGATT, and NAAAC, wherein N is any nucleotide, W is A or T, and R is A or G. At the typical length, only about 5-7% of the target sites would be unique within a target genome, indicating that off target effects could be significant. The length of the target site can be expanded by requiring two binding events. For example, CRISPR-based endonucleases can be modified such that they can only cleave one strand of a double-stranded sequence (i.e., converted to nickases). Thus, the use of a pair of CRISPR-based nickases in combination with two different guide RNAs would essentially double the length of the target site, while still effecting a double stranded break.

In some embodiments, therefore, the Cas9-derived endonuclease can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the domain lacks nuclease activity). In some embodiments in which one of the nuclease domains is inactive, the Cas9-derived protein is able to introduce a nick into a double-stranded nucleic acid (such protein is termed a “nickase”), but not cleave the double-stranded DNA. For example, an aspartate to alanine (D10A) conversion in a RuvC-like domain converts the Cas9-derived protein into a “HNH” nickase. Likewise, a histidine to alanine (H840A) conversion (in some instances, the histidine is located at position 839) in a HNH domain converts the Cas9-derived protein into a “RuvC” nickase. Thus, for example, in one embodiment the Cas9-derived nickase has an aspartate to alanine (D10A) conversion in a RuvC-like domain. In another embodiment, the Cas9-derived nickase has a histidine to alanine (H840A or H839A) conversion in a HNH domain. The RuvC-like or HNH-like nuclease domains of the Cas9-derived nickase can be modified using well-known methods, such as site-directed mutagenesis, PCR-mediated mutagenesis, and total gene synthesis, as well as other methods known in the art. In further embodiments, a pair to Cas9-derived nickases can be used in combination to create a double-stranded break in the chromosomal sequence of interest.

Additional domains. The CRISPR/Cas endonuclease or nickase generally comprises at least one nuclear localization signal (NLS). For example, in one embodiment, the NLS can be a monopartite sequence, such as PKKKRKV (SEQ ID NO: 1) or PKKKRRV (SEQ ID NO: 2). In another embodiment, the NLS can be a bipartite sequence. In still another embodiment, the NLS can be KRPAATKKAGQAKKKK (SEQ ID NO: 3). The NLS can be located at the N-terminus, the C-terminus, or in an internal location of the protein.

In some embodiments, the CRISPR/Cas endonuclease or nickase can further comprise at least one cell-penetrating domain. The cell-penetrating domain can be a cell-penetrating peptide sequence derived from the HIV-1 TAT protein. As an example, the TAT cell-penetrating sequence can be GRKKRRQRRRPPQPKKKRKV (SEQ ID NO: 4). In another embodiment, the cell-penetrating domain can be TLM (PLSSIFSRIGDPPKKKRKV; SEQ ID NO: 5), a cell-penetrating peptide sequence derived from the human hepatitis B virus. In still another embodiment, the cell-penetrating domain can be MPG (GALFLGWLGAAGSTMGAPKKKRKV; SEQ ID NO: 6 or GALFLGFLGAAGSTMGAWSQPKKKRKV; SEQ ID NO: 7). In an additional embodiment, the cell-penetrating domain can be Pep-1 (KETWWETWWTEWSQPKKKRKV; SEQ ID NO: 8), VP22, a cell penetrating peptide from Herpes simplex virus, or a polyarginine peptide sequence. The cell-penetrating domain can be located at the N-terminus, the C-terminus, or in an internal location of the protein.

In still other embodiments, the CRISPR/Cas endonuclease or nickase can further comprise at least one marker domain. Non-limiting examples of marker domains include fluorescent proteins, purification tags, and epitope tags. In one embodiment, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalamal, GFPuv, Sapphire, T-sapphire), cyan fluorescent proteins (e.g. ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In another embodiment, the marker domain can be a purification tag and/or an epitope tag. Suitable tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, 51, T7, V5, VSV-G, 6×His, biotin carboxyl carrier protein (BCCP), and calmodulin. The marker domain can be located at the N-terminus, the C-terminus, or in an internal location of the protein. The marker domain can be linked to the CRISPR/Cas endonuclease or nickase by a 2A peptide (Szymczak et al., 2004, Nat. Biotechnol., 589(5):589-94).

Guide RNA.

The CRISPR/Cas endonuclease is guided to the targeted site by a guide RNA. A guide RNA interacts with both the CRISPR/Cas endonuclease and the target site in the chromosomal, at which site the CRISPR/Cas endonuclease or nickase cleaves at least one strand of the double-stranded sequence. The guide RNA can be introduced into the cell along with CRISPR/Cas endonuclease or nucleic acid encoding the CRISPR/Cas endonuclease. Alternatively, DNA encoding both the CRISPR/Cas endonuclease and the guide RNA can be introduced into the cell.

A guide RNA comprises three regions: a first region at the 5′ end that is complementary to sequence at the target site, a second internal region that forms a stem loop structure, and a third 3′ region that remains essentially single-stranded. The first region of each guide RNA is different such that each guide RNA guides a CRISPR/Cas endonuclease or nickase to a specific target site. The second and third regions (also called the scaffold region) of each guide RNA can be the same in all guide RNAs.

The first region of the guide RNA is complementary to sequence (i.e., protospacer sequence) at the target site such that the first region of the guide RNA can base pair with sequence at the target site. In general, there are no mismatches between the sequence of the first region of the guide RNA and the sequence at the target site (i.e., the complementarity is total). In various embodiments, the first region of the guide RNA can comprise from about 10 nucleotides to more than about 25 nucleotides. For example, the region of base pairing between the first region of the guide RNA and the target site in the chromosomal sequence can be about 10, 11, 12, 13, 14, 15, 16, 17, 18, 19, 20, 22, 23, 24, 25, or more than 25 nucleotides in length. In exemplary embodiments, the first region of the guide RNA is about 19 or 20 nucleotides in length.

The guide RNA also comprises a second region that forms a secondary structure. In some embodiments, the secondary structure comprises a stem (or hairpin) and a loop. The length of the loop and the stem can vary. For example, the loop can range from about 3 to about 10 nucleotides in length, and the stem can range from about 6 to about 20 base pairs in length. The stem can comprise one or more bulges of 1 to about 10 nucleotides. Thus, the overall length of the second region can range from about 16 to about 60 nucleotides in length. In an exemplary embodiment, the loop is about 4 nucleotides in length and the stem comprises about 12 base pairs.

The guide RNA also comprises a third region at the 3′ end that remains essentially single-stranded. Thus, the third region has no complementarity to any chromosomal sequence in the cell of interest and has no complementarity to the rest of the guide RNA. The length of the third region can vary. In general, the third region is more than about 4 nucleotides in length. For example, the length of the third region can range from about 5 to about 60 nucleotides in length.

The combined length of the second and third regions (or scaffold) of the guide RNA can range from about 30 to about 120 nucleotides in length. In one aspect, the combined length of the second and third regions of the guide RNA range from about 70 to about 100 nucleotides in length.

In some embodiments, the guide RNA comprises one molecule comprising all three regions. In other embodiments, the guide RNA can comprise two separate molecules. The first RNA molecule can comprise the first region of the guide RNA and one half of the “stem” of the second region of the guide RNA. The second RNA molecule can comprise the other half of the “stem” of the second region of the guide RNA and the third region of the guide RNA. Thus, in this embodiment, the first and second RNA molecules each contain a sequence of nucleotides that are complementary to one another. For example, in one embodiment, the first and second RNA molecules each comprise a sequence (of about 6 to about 20 nucleotides) that base pairs to the other sequence to form a functional guide RNA.

(iii) Other Targeting Endonucleases

In further embodiments, the targeting endonuclease can be a meganuclease. Meganucleases are endodeoxyribonucleases characterized by long recognition sequences, i.e., the recognition sequence generally ranges from about 12 base pairs to about 40 base pairs. As a consequence of this requirement, the recognition sequence generally occurs only once in any given genome. Among meganucleases, the family of homing endonucleases named LAGLIDADG has become a valuable tool for the study of genomes and genome engineering (see, e.g., Arnould et al., 2011, Protein Eng Des Sel, 24(1-2):27-31). Other suitable meganucleases include I-Crel and I-Dmol. A meganuclease can be targeted to a specific chromosomal sequence by modifying its recognition sequence using techniques well known to those skilled in the art.

In additional embodiments, the targeting endonuclease can be a transcription activator-like effector (TALE) nuclease. TALEs are transcription factors from the plant pathogen Xanthomonas that can be readily engineered to bind new DNA targets. TALEs or truncated versions thereof may be linked to the catalytic domain of endonucleases such as Fokl to create targeting endonuclease called TALE nucleases or TALENs (Sanjana et al., 2012, Nat Protoc, 7(1):171-192) and Arnould et al., 2011, Protein Engineering, Design & Selection, 24(1-2):27-31).

In alternate embodiments, the targeting endonuclease can be chimeric nuclease. Non-limiting examples of chimeric nucleases include ZF-meganucleases, TAL-meganucleases, Cas9-Fokl fusions, ZF-Cas9 fusions, TAL-Cas9 fusions, and the like. Persons skilled in the art are familiar with means for generating such chimeric nuclease fusions.

In still other embodiments, the targeting endonuclease can be a site-specific endonuclease. In particular, the site-specific endonuclease can be a “rare-cutter” endonuclease whose recognition sequence occurs rarely in a genome. Alternatively, the site-specific endonuclease can be engineered to cleave a site of interest (Friedhoff et al., 2007, Methods Mol Biol 352:1110123). Generally, the recognition sequence of the site-specific endonuclease occurs only once in a genome. In alternate further embodiments, the targeting endonuclease can be an artificial targeted DNA double strand break inducing agent.

(iv) Optional Polynucleotide

The method for targeted genome modification or engineering can further comprise introducing into the cell at least one polynucleotide comprising a sequence having substantial sequence identity to a sequence on at least one side of the targeted cleavage site such that the double-stranded break introduced by the targeting endonuclease can be repaired by a homology-directed repair process and the sequence of the polynucleotide is exchanged with the endogenous chromosomal sequence, thereby modifying the endogenous chromosomal sequence. For example, the polynucleotide comprises a first sequence having substantial sequence identity to sequence on one side of the targeted cleavage site and a second sequence having substantial sequence identity to sequence on the other side of the targeted cleavage site. Alternatively, the polynucleotide comprises a first sequence having substantial sequence identity to sequence on one side of the targeted cleavage site and a second sequence having substantial sequence identity to a sequence located away from the targeted cleavage site. The sequence located away from the targeted cleavage site may be tens, hundreds, or thousands of nucleotides upstream or downstream of the targeted cleavage site. The polynucleotide may further comprise a donor sequence for integration into the targeted chromosomal sequence. For example, the donor sequence can be an exogenous sequence encoding a protein of interest. Alternatively, the donor sequence can be an exogenous promoter control sequence or enhancer element.

The lengths of the first and second sequences in the polynucleotide that have substantial sequence identity to sequences in the targeted chromosomal sequence can and will vary. In general, each of the first and second sequences in the polynucleotide is at least about 10 nucleotides in length. In various embodiments, the polynucleotide sequences having substantial sequence identity with chromosomal sequences can be about 15 nucleotides, about 20 nucleotides, about 25 nucleotides, about 30 nucleotides, about 40 nucleotides, about 50 nucleotides, about 100 nucleotides, or more than 100 nucleotides in length.

The phrase “substantial sequence identity” means that the sequences in the polynucleotide have at least about 75% sequence identity with the chromosomal sequences of interest. In some embodiments, the sequences in the polynucleotide about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the chromosomal sequences of interest.

The length of the polynucleotide can and will vary. For example, the polynucleotide can range from about 20 nucleotides in length up to about 200,000 nucleotides in length. In various embodiments, the polynucleotide ranges from about 20 nucleotides to about 100 nucleotides in length, from about 100 nucleotides to about 1000 nucleotides in length, from about 1000 nucleotides to about 10,000 nucleotides in length, from about 10,000 nucleotides to about 100,000 nucleotides in length, or from about 100,000 nucleotides to about 200,000 nucleotides in length.

Typically, the polynucleotide is DNA. The DNA can be single-stranded or double-stranded. The polynucleotide can be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. In certain embodiments, the polynucleotide is single-stranded. In exemplary embodiments, the polynucleotide is a single-stranded oligonucleotide comprising less than about 200 nucleotides.

In some embodiments, the polynucleotide further comprises a marker. Such a marker may enable screening for targeted integrations. In some embodiments, the marker is a restriction endonuclease site. In other embodiments the marker is a fluorescent protein, a purification tag, or an epitope tag. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalamal, GFPuv, Sapphire, T-sapphire), cyan fluorescent proteins (e.g. ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker can be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, 51, T7, V5, VSV-G, 6×His, biotin carboxyl carrier protein (BCCP), and calmodulin.

(v) Delivery to the Cell

The method comprises introducing the targeting endonuclease into the cell of interest. The targeting endonuclease can be introduced into the cell as a purified isolated protein or as a nucleic acid encoding the targeting endonuclease. The nucleic acid may be DNA or RNA. In embodiments in which the encoding nucleic acid is mRNA, the mRNA may be 5′ capped and/or 3′ polyadenylated. In embodiments in which the encoding nucleic acid is DNA, the DNA may be linear or circular. The DNA may be part of a vector, wherein the encoding DNA may be operably linked to a suitable promoter. Those skilled in the art are familiar with appropriate vectors, promoters, other control elements, and means of introducing the vector into the cell of interest.

The targeting endonuclease molecule(s) and the optional polynucleotide(s) described above can be introduced into the cell by a variety of means. Suitable delivery means include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In a specific embodiment, the targeting endonuclease molecule(s) and polynucleotides(s) are introduced into the cell by nucleofection.

In embodiments in which more than one targeting endonuclease molecule and more than one polynucleotide are introduced into a cell, the molecules can be introduced simultaneously or sequentially. For example, targeting endonuclease molecules, each specific for a targeted cleavage site (and optional polynucleotides) can be introduced at the same time. Alternatively, each targeting endonuclease molecule, as well as the optional polynucleotides(s) can be introduced sequentially.

The ratio of the targeting endonuclease molecule(s) to the optional polynucleotide(s) can and will vary. In general, the ratio of targeting endonuclease molecule(s) to polynucleotide(s) ranges from about 1:10 to about 10:1. In various embodiments, the ratio of the targeting endonuclease molecule(s) to polynucleotide(s) may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio is about 1:1.

(vi) Culturing the Cell

The method further comprises maintaining the cell under appropriate conditions such that the double-stranded break introduced by the targeting endonuclease can be repaired by (i) a non-homologous end-joining repair process such that the chromosomal sequence is modified by a deletion, insertion and/or substitution of at least one nucleotide or, optionally, (ii) a homology-directed repair process such that the chromosomal sequence is exchanged with the sequence of the polynucleotide such that the chromosomal sequence is modified. In embodiments in which nucleic acid(s) encoding the targeting endonuclease(s) is introduced into the cell, the method comprises maintaining the cell under appropriate conditions such that the cell expresses the targeting endonuclease(s).

In general, the cell is maintained under conditions appropriate for cell growth and/or maintenance. Suitable cell culture conditions are well known in the art and are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

During this step of the process, the targeting endonuclease(s) recognizes, binds, and creates a double-stranded break(s) at the targeted cleavage site(s) in the chromosomal sequence, and during repair of the double-stranded break(s) a deletion, insertion, and/or substitution of at least one nucleotide is introduced into the targeted chromosomal sequence. In specific embodiments, the targeted chromosomal sequence is inactivated.

Upon confirmation that the chromosomal sequence of interest has been modified, single cell clones can be isolated and genotyped (via DNA sequencing and/or protein analyses). Cells comprising one modified chromosomal sequence can undergo one or more additional rounds of targeted genome modification to modify additional chromosomal sequences.

(b) RNA Interference

In other embodiments, the viral resistant cell line can be prepared using an RNA interference (RNAi) agent that inhibits expression of a target mRNA or transcript. The RNAi agent can lead to cleavage of the target mRNA or transcript. Alternatively, the RNAi agent can prevent or disrupt translation of the target mRNA into protein.

In some embodiments, the RNAi agent can be a short interfering RNA (siRNA). In general, a siRNA comprises a double-stranded RNA molecule that ranges from about 15 to about 29 nucleotides in length. The siRNA can be about 16-18, 17-19, 21-23, 24-27, or 27-29 nucleotides in length. In a specific embodiment, the siRNA is about 21 nucleotides in length. The siRNA can optionally further comprise one or two single-stranded overhangs, e.g., a 3′ overhang on one or both ends. The siRNA can be formed from two RNA molecules that hybridize together or, alternatively, can be generated from a short hairpin RNA (shRNA) (see below). In some embodiments, the two strands of the siRNA are completely complementary, such that no mismatches or bulges exist in the duplex formed between the two sequences. In other embodiments, the two strands of the siRNA are substantially complementary, such that one or more mismatches and/or bulges may exist in the duplex formed between the two sequences. In certain embodiments, one or both of the 5′ ends of the siRNA have a phosphate group, while in other embodiments one or both of the 5′ ends lack a phosphate group. In other embodiments, one or both of the 3′ ends of the siRNA have a hydroxyl group, while in other embodiments one or both of the 5′ ends lack a hydroxyl group.

One strand of the siRNA, which is referred to as the “antisense strand” or “guide strand,” includes a portion that hybridizes with the target transcript. In certain embodiments, the antisense strand of the siRNA is completely complementary with a region of the target transcript, i.e., it hybridizes to the target transcript without a single mismatch or bulge over a target region between about 15 and about 29 nucleotides in length, preferably at least 16 nucleotides in length, and more preferably about 18-20 nucleotides in length. In other embodiments, the antisense strand is substantially complementary to the target region, i.e., one or more mismatches and/or bulges may exist in the duplex formed by the antisense strand and the target transcript. Typically, siRNAs are targeted to exonic sequences of the target transcript. Those of skill in the art are familiar with programs, algorithms, and/or commercial services that design siRNAs for target transcripts. An exemplary example is the Rosetta siRNA Design Algorithm (Rosetta Inpharmatics, North Seattle, Wash.) and MISSION® siRNA (Sigma-Aldrich, St. Louis, Mo.). The siRNA can be enzymatically synthesized in vitro using methods well known to those of skill in the art. Alternatively, the siRNA can be chemically synthesized using oligonucleotide synthesis techniques that are well known in the art.

In other embodiments, the RNAi agent can be a short hairpin RNA (shRNA). In general, a shRNA is an RNA molecule comprising at least two complementary portions that are hybridized or are capable of hybridizing to form a double-stranded structure sufficiently long to mediate RNA interference (as described above), and at least one single-stranded portion that forms a loop connecting the regions of the shRNA that form the duplex. The structure is also called a stem-loop structure, with the stem being the duplex portion. In some embodiments, the duplex portion of the structure is completely complementary, such that no mismatches or bulges exist in the duplex region of the shRNA. In other embodiments, the duplex portion of the structure is substantially complementary, such that one or more mismatches and/or bulges exist in the duplex portion of the shRNA. The loop of the structure can be from about 1 to about 20 nucleotides in length, preferably from about 4 to about 10 about nucleotides in length, and more preferably from about 6 to about 9 nucleotides in length. The loop can be located at either the 5′ or 3′ end of the region that is complementary to the target transcript (i.e., the antisense portion of the shRNA).

The shRNA can further comprise an overhang on the 5′ or 3′ end. The optional overhang can be from about 1 to about 20 nucleotides in length, and more preferably from about 2 to about 15 nucleotides in length. In some embodiments, the overhang comprises one or more U residues, e.g., between about 1 and about 5 U residues. In some embodiments, the 5′ end of the shRNA has a phosphate group, while in other embodiments it does not. In other embodiments, the 3′ end of the shRNA has a hydroxyl group, while in other embodiments it does not. In general, shRNAs are processed into siRNAs by the conserved cellular RNAi machinery. Thus, shRNAs are precursors of siRNAs and are similarly capable of inhibiting expression of a target transcript that is complementary to a portion of the shRNA (i.e., the antisense portion of the shRNA). Those of skill in the art are familiar with the available resources (as detailed above) for the design and synthesis of shRNAs.

In still other embodiments, the RNAi agent can be an RNAi expression vector. Typically, an RNAi expression vector is used for intracellular (in vivo) synthesis of RNAi agents, such as siRNAs or shRNAs. In one embodiment, two separate, complementary siRNA strands are transcribed using a single vector containing two promoters, each of which directs transcription of a single siRNA strand (i.e., each promoter is operably linked to a template for the siRNA so that transcription may occur). The two promoters can be in the same orientation, in which case each is operably linked to a template for one of the complementary siRNA strands. Alternatively, the two promoters can be in opposite orientations, flanking a single template so that transcription for the promoters results in synthesis of two complementary siRNA strands. In another embodiment, the RNAi expression vector can contain a promoter that drives transcription of a single RNA molecule comprising two complementary regions, such that the transcript forms a shRNA.

Those of skill in the art will appreciate that it is preferable for siRNA and shRNA agents to be produced in vivo via the transcription of more than one transcription unit. Generally speaking, the promoters utilized to direct in vivo expression of the one or more siRNA or shRNA transcription units may be promoters for RNA polymerase III (Pol III). Certain Pol III promoters, such as U6 or H1 promoters, do not require cis-acting regulatory elements within the transcribed region, and thus, are preferred in certain embodiments. In other embodiments, promoters for Pol II can be used to drive expression of the one or more siRNA or shRNA transcription units. In some embodiments, tissue-specific, cell-specific, or inducible Pol II promoters can be used.

A construct that provides a template for the synthesis of siRNA or shRNA can be produced using standard recombinant DNA methods and inserted into any of a wide variety of different vectors suitable for expression in eukaryotic cells. Recombinant DNA techniques are described in Ausubel et al, 2003, supra and Sambrook & Russell, 2001, supra. Those of skill in the art also appreciate that vectors can comprise additional regulatory sequences (e.g., termination sequence, translational control sequence, etc.), as well selectable marker sequences. DNA plasmids are known in the art, including those based on pBR322, PUC, and so forth. Since many expression vectors already contain a suitable promoter or promoters, it may be only necessary to insert the nucleic acid sequence that encodes the RNAi agent of interest at an appropriate location with respect to the promoter(s). Viral vectors can also be used to provide intracellular expression of RNAi agents. Suitable viral vectors include retroviral vectors, lentiviral vectors, adenoviral vectors, adeno-associated virus vectors, herpes virus vectors, and so forth. In a specific embodiment, the RNAi expression vector is a shRNA lentiviral-based vector or lentiviral particle, such as that provided in MISSION® TRC shRNA products (Sigma-Aldrich).

The RNAi agents or RNAi expression vectors can be introduced into the cell using methods well known to those of skill in the art. Such techniques are described in Ausubel et al., 2003, supra or Sambrook & Russell, 2001, supra, for example. In certain embodiments, the RNAi expression vector, e.g., a viral vector, is stably integrated into the genome of the cell, such that expression of the target gene is disrupted over subsequent cell generations.

(c) Site-Specific Recombination

In alternate embodiments, the viral resistance cell lines can be prepared using site-specific recombination techniques. For example, site-specific recombination techniques can be used to delete all or part of a chromosomal sequence of interest, or introduce single nucleotide polymorphisms (SNPs) into the chromosomal sequence of interest. In one embodiment, the chromosomal sequence of interest is targeted using a Cre-loxP site-specific recombination system, a Flp-FRT site-specific recombination system, or variants thereof. Such recombination systems are commercially available, and additional teaching for these techniques is found in Ausubel et al., 2003, supra, for example.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, “deficient” refers to reduced or non-detectable levels of the targeted enzymes or proteins, or reduced or non-detectable activity of the targeted enzymes or proteins.

As used herein, the term “endogenous sequence” refers to a chromosomal sequence that is native to the cell.

The term “exogenous sequence” refers to a chromosomal sequence that is not native to the cell, or a chromosomal sequence that is moved to a different chromosomal location.

A “genetically modified” cell refers to a cell in which the genome has been modified or engineered, i.e., the cell contains at least chromosomal sequence that has been engineered to contain an insertion of at least one nucleotide, a deletion of at least one nucleotide, and/or a substitution of at least one nucleotide.

The terms “genome modification” and “genome editing” refer to processes by which a specific chromosomal sequence is changed such that the chromosomal sequence is modified. The chromosomal sequence may be modified to comprise an insertion of at least one nucleotide, a deletion of at least one nucleotide, and/or a substitution of at least one nucleotide. The modified chromosomal sequence is inactivated such that no product is made. Alternatively, the chromosomal sequence can be modified such that an altered product is made.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms can encompass known analogs of natural nucleotides, as well as nucleotides that are modified in the base, sugar and/or phosphate moieties. In general, an analog of a particular nucleotide has the same base-pairing specificity; i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site” or “target sequence” refer to a nucleic acid sequence that defines a portion of a chromosomal sequence to be modified or edited and to which a targeting endonuclease is engineered to recognize and bind, provided sufficient conditions for binding exist.

The terms “upstream” and “downstream” refer to locations in a nucleic acid sequence relative to a fixed position. Upstream refers to the region that is 5′ (i.e., near the 5′ end of the strand) to the position and downstream refers to the region that is 3′ (i.e., near the 3′ end of the strand) to the position.

As used herein, “viral resistance” refers to the ability of cells to resist viral infection. More specifically, entry of a virus and/or propagation of a virus is reduced or eliminated in the engineered cell lines disclosed herein as compared to unmodified parental cell lines.

The term “virus,” as used herein refers to virus particles (i.e., virions) and parts thereof (e.g., capsid shell, inner core of nucleic acid, etc.).

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

As various changes could be made in the above-described cells and methods without departing from the scope of the invention, it is intended that all matter contained in the above description and in the examples given below, shall be interpreted as illustrative and not in a limiting sense.

### EXAMPLES

The following examples illustrate certain aspects of the invention.

### Example 1: Preparation of Knockout CHO Cell Lines

ZFN-mediated gene modification techniques were employed to inactivate (i.e., knock out) genes encoding proteins of interest. The genes targeted included dynamin-1, dynamin-2, dynamin-3, integrin beta 1, and integrin alpha 5. For this, pairs of ZFNs targeting specific sites within the coding region of the genes of interest were designed using a proprietary algorithm. ZFN expression constructs were prepared using standard procedures. ZFN mRNA was produced from ZFN plasmid DNA using standard in vitro transcription, mRNA poly-adenylation, capping, and purification methods. Parental cells were maintained as suspension cultures in appropriate growth media, and cells were seeded at 0.5×10⁶ cells/mL in bioreactor tubes one day prior to transfection. Typically, each transfection contained 1×10⁶ cells in 150 μL growth media and 5 μg ZFN DNA or mRNA. Transfections were conducted by electroporation at 140 V and 950 μF in 0.2 cm cuvettes. Electroporated cells were placed in 2 mL growth media in a 6-well plate static culture.

On days 3 and 10 post-transfection, cells were removed and genomic DNA was isolated using a genomic DNA miniprep kit (Sigma-Aldrich). ZFN-induced cleavage was verified using a Cel-1 nuclease assay, as described in CompoZr® Knockout ZFN product information. This assay determines the efficiency of ZFN-mediated gene mutation as described previously (Miller et al., Nat. Biotechnol. 2007, 25:778-785). The assay detects alleles of the targeted locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of wild type (WT) and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis.

Upon confirmation of ZFN activity, the ZFN transfected cells were single-cell cloned using limiting dilution. For this, cells were plated at an approximate density of about 0.5 cell/well using a mixture of 80% CHO serum-free cloning media, 20% conditioned media, and 4 mM L-glutamine. Clonality and growth were microscopically verified on days 7 and 14 post plating, respectively. Clones with growth were be expanded and genotyped by PCR and/or DNA sequencing. Some of the dynamin KO clones underwent one or more further rounds of ZFN-mediated gene modification to generate double knockout (DKO) or triple knockout (TKO) cells/clones.

### Example 2: Viral Infections and Viral Resistance Testing Assays

The dynamin DKO and the integrin KO clones were then tested for their ability to support or resist infection following challenge with the prototype MVM virus (strain MVMp). Briefly, cells were grown in the appropriate media and MVMp virus was added at a suitable multiplicity of infection (MOI). Control cells were wild type CHO cells. At 0 and 21 hours post infection, cells were harvested by centrifugation, and levels of viral DNA were estimated via PCR.

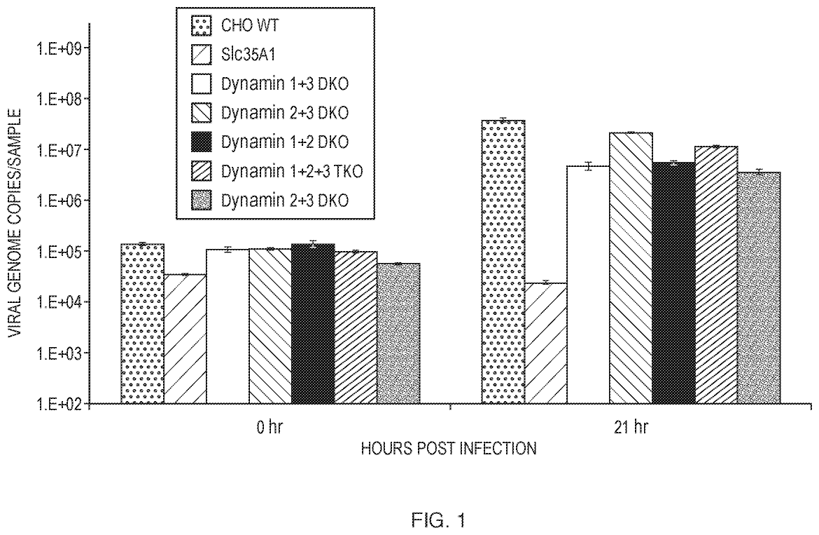

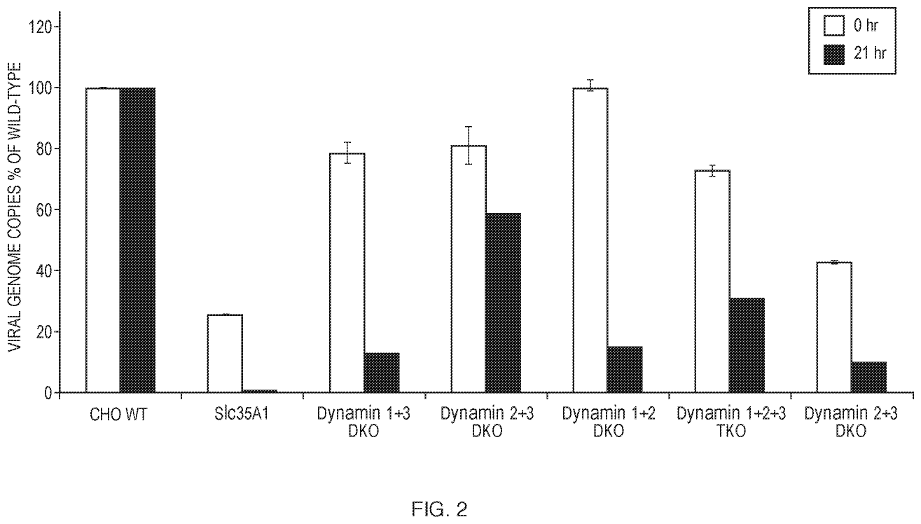

FIGS. 1 and 2 show the levels of resistance to MVM infection of the dynamin DKO clones. While all of the dynamin DKO clones showed reduced viral content at 21 hr (see FIG. 1), the effect is more pronounced with the levels are normalized to the wild type control cells (see FIG. 2).

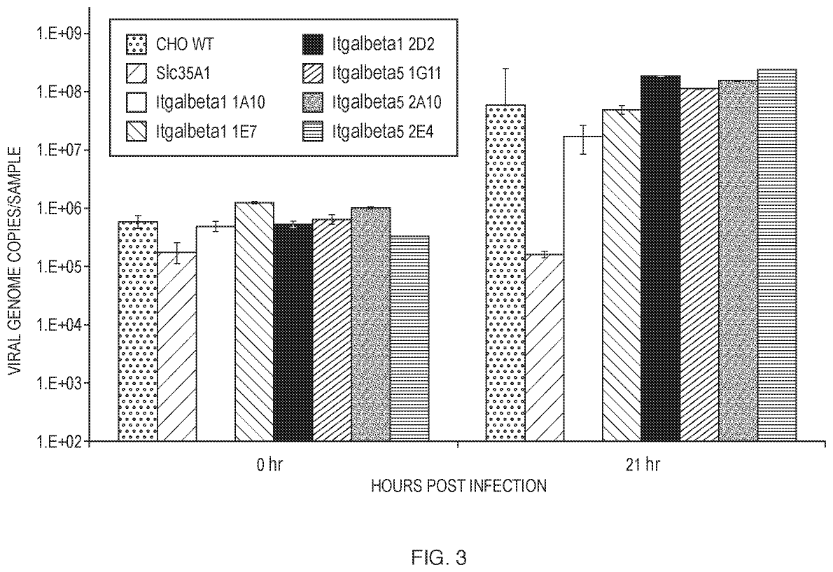

FIG. 3 presents the levels of resistance to MVM infection of the integrin KO clones. Two of the three beta 1 clones showed reduced viral levels at 21 hrs, but the other beta 1 and alpha 5 clones did not.

1. A mammalian cell line engineered to have reduced expression of a protein chosen from integrin, beta 1; integrin, beta 2; integrin, beta 3; integrin, beta 4; integrin, beta 5; integrin, beta 6; integrin, beta 7; integrin, beta 8; integrin, alpha 1; integrin, alpha 2; integrin, alpha 3; integrin, alpha 4; integrin, alpha 5; integrin, alpha 6; integrin, alpha 7; integrin, alpha 8; integrin, alpha 9; integrin, alpha 10; integrin, alpha 11, integrin, alpha D, integrin, alpha E; integrin, alpha L; integrin, alpha V; integrin, alpha 2B; integrin, alpha X; talin 1; talin 2; xylosyltransferase 1; xylosyltransferase 2; β4-galactosyltransferase; β3-galactosyltransferase; β3-GlcA transferase; exostosin 1; exostosin 2; exostosin-like 1; exostosin-like 2; exostosin-like 3; bifunctional heparan sulfate N-deacetylase/N-sulfotransferase 1; D-glucuronyl C5-epimerase; heparan sulfate 2-O-sulfotransferase 1; heparan sulfate 6-O-sulfotransferase 1; heparan sulfate 3-O sulfotransferase; carbohydrate (N-acetylgalactosamine 4-O) sulfotransferase 8; carbohydrate (chondroitin 6) sulfotransferase 3; carbohydrate (N-acetylglucosamine 6-O) sulfotransferase 7; carbohydrate (N-acetylgalactosamine 4-sulfate 6-O) sulfotransferase 15; carbohydrate (N-acetylglucosamine 6-O sulfotransferase 5; hyaluronan synthase protein 1; hyaluronan synthase protein 2; hyaluronan synthase protein 3; dynamin-1; dynamin-2; dynamin-3; caveolin 1; cell division cycle 42; ADP-ribosylation factor 6; Ras-related C3 botulinum toxin substrate 1; lysosome-associated membrane glycoprotein 1; chloride channel, voltage-sensitive 1; chloride channel, voltage-sensitive 2; H(+)/Cl(−) exchange transporter 3; H(+)/Cl(−) exchange transporter 4; H(+)/Cl(−) exchange transporter 5; H(+)/Cl(−) exchange transporter 6; H(+)/Cl(−) exchange transporter 7; V-type proton ATPase catalytic subunit A; ATPase, H+ transporting, lysosomal 70 kDa, V1 subunit 131; ATPase, H+ transporting, lysosomal 70 kDa, V1 subunit B2; ATPase, H+ transporting, lysosomal accessory Protein 1 (Ac45); ATPase, H+ transporting, lysosomal 42 kDa, V1 subunit C2; serine-protein kinase ATM; RAF proto-oncogene serine/threonine-protein kinase; ATM serine/threonine kinase; bromodomain adjacent to zinc finger domain 1B; casein kinase 2, alpha 1 polypeptide; casein kinase II subunit alpha; cofilin-1; cofilin-2; exportin-1; amyloid beta (A4) precursor protein-binding, family B, member 1 interacting protein; phosphatidylinositol-4-phosphate 5-kinase, type I, gamma, or combination thereof.

2. The mammalian cell line of claim 1, wherein expression of the protein is reduced due to inactivation of at least one chromosomal sequence encoding the protein.

3. The mammalian cell line of claim 2, wherein the chromosomal sequence is inactivated using a targeting endonuclease-mediated genome modification technique.

4. The mammalian cell line of claim 3, wherein the targeting endonuclease is a zinc finger nuclease or a CRISPR/Cas endonuclease.

5. The mammalian cell line of claim 2, wherein expression of the protein is eliminated due to inactivation of all chromosomal sequences encoding the protein.

6. The mammalian cell line of claim 2 further comprising reduced expression of galectin-3, vimentin, caspase 3, gelsolin, WD repeat containing protein 1 (Wdr1), radixin, moesin, core 1 enzyme chaperone (COSMC), solute carrier family 35 (CMP-sialic acid transporter) member A1 (Slc35A1), core 1 elongation enzyme (C1GalT1), St3 beta-galactoside alpha-2,3-sialyltransferase 1 (St3Gal1), St3 beta-galactoside alpha-2,3-sialyltransferase 2 (St3Gal2), St3 beta-galactoside alpha-2,3-sialyltransferase 3 (St3Gal3), St3 beta-galactoside alpha-2,3-sialyltransferase 4 (St3Gal4), St3 beta-galactoside alpha-2,3-sialyltransferase 5 (St3Gal5), St3 beta-galactoside alpha-2,3-sialyltransferase 6 (St3Gal6), or combination thereof.

7. The mammalian cell line of claim 2 further comprising increased expression of promyelocytic leukemia protein (PML) and/or expression of a dominant negative form of a viral protein chosen from NS1, NS2, VP1, VP2, or combination thereof.

8. The mammalian cell line of claim 7, wherein the virus is a member of Parvoviridae, Reoviridae, Caliciviridae, Paramyxoviridae, Coronaviridae, Picornaviridae, Polyomaviridae, Bunyaviridae, or combination thereof.

9. The mammalian cell line of claim 8, wherein the virus is a parvovirus chosen from minute virus of mouse (MVM), mouse parvovirus type-1, mouse parvovirus type-2, mouse parvovirus type-3, porcine parvovirus 1, bovine parovirus 1, human parovirus B19, human parovirus 4, human parovirus 5, or combination thereof.

10. The mammalian cell line of claim 8, wherein the virus is a reovirus chosen from mammalian reovirus-3, mammalian orthoreovirus, avian orthoreovirus, or combination thereof.

11. The mammalian cell line of claim 2, further comprising at least one nucleic acid encoding a recombinant protein chosen from an antibody, an antibody fragment, a vaccine, a growth factor, a cytokine, a hormone, or a clotting factor.

12. The mammalian cell line of claim 2, wherein the cell line is a non-human cell line.

13. The mammalian cell line of claim 12, wherein the cell line is a Chinese hamster ovary (CHO) cell line.

14. The mammalian cell line of claim 12, for use in a biologic production system.

15. A cell culture system comprising a Selective Inhibitor of Nuclear Export (SINE), a sialic acid analog, a small molecule inhibitor of CMP sialic acid transporter, a sialidase, a neuraminidase, or combination thereof, and a cell growth medium.

16. The cell culture system of claim 15, wherein the SINE is chosen from leptomycin B, ratjadone, goniothalamin, N-azolylacrylates, anguinomycin, CBS9106, selinexor (KPT-330), verdinexor (KPT-335), KPT-185, KPT-251, KPT-276, or combination thereof.

17. The cell culture system of claim 15, wherein the sialic acid analog is chosen from P-3Fₐₓ-Neu5AC, oseltamivir, zanamivir, 5-acteylneuraminic acid derivates, 2-alpha-O-methyl-5-acetylneuraminic acid, or combination thereof.

18. The cell culture system of claim 15, wherein the small molecule inhibitor of CMP sialic acid transporter is chosen from KI-8110, 2′-O-methyl CMP, 5-methyl CMP, or combination thereof.

19. The cell culture system of claim 15, wherein the cell growth medium is animal component free.

20. The cell culture system of claim 15, which is used in combination with the mammalian cell line of claim 12 to produce a recombinant protein.

21. A composition comprising the mammalian cell line of claim 12 and at least one virus, wherein the cell line exhibits resistance to infection by the virus.

22. The composition of claim 21, further comprising the cell culture system of claim 20.

23. A method for reducing the risk of viral contamination of a biologic production system, the method comprising providing for use in the biologic production system the mammalian cell line of claim 12 and/or the cell culture system of claim 15.
